(19) United States
(12) Patent Application Publication
(10) Pub. No.: US 2004/0063688 A1
(43) Pub. Date: Apr. 1, 2004

(54) Novel piperidine derivatives

(75) Inventors: Laura C. Blumberg, Waterford, CT (US); Matthew F. Brown, Stonington, CT (US); Matthew M. Hayward, Old Lyme, CT (US); Christopher S. Poss, Baltic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/616,843

(22) Filed: Jul. 8, 2003

Related U.S. Application Data
(60) Provisional application No. 60/397,263, filed on Jul. 18, 2002.

(51) Int. Cl. C07D 265/36; C07D 498/02; C07D 451/02; A61K 31/55; A61K 31/538; A61K 31/46; A61K 31/403

(52) U.S. Cl. 514/216; 514/230.5; 514/304; 514/412; 540/593; 544/105; 546/124; 548/453

(57) ABSTRACT

A compound of the formula

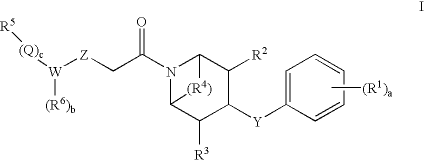

wherein a, b, c R¹, R², R³, R⁴, R⁵, R⁶, Q, W, Y, and Z are defined as above, useful as potent and selective inhibitors of MIP-1α (CCL3) binding to its receptor CCR1 found on inflammatory and immunomodulatory cells (preferably leukocytes and lymphocytes).

This application claims the benefit of priority of U.S. provisional Patent Application Serial No. 60/397,263 filed Jul. 18, 2002, which is incorporated herein in its entirety for all purposes.

### BACKGROUND OF THE INVENTION

The present invention relates to novel piperidine derivatives, methods of use and pharmaceutical compositions containing them.

The compounds of the invention are potent and selective inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment or prevention of autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. Compounds of this invention are also potentially useful for the treatment or prevention of cancer metastasis, including but not limited to breast cancer. Compounds of this invention may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Compounds of this invention may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

MIP-1α and RANTES are soluble chemotactic peptides (chemokines) which are produced by inflammatory cells, in particular CD8+ lymphocytes, polymorphonuclear leukocytes (PMNs) and macrophages, J. Biol. Chem., 270 (30) 29671-29675 (1995). These chemokines act by inducing the migration and activation of key inflammatory and immunomodulatory cells. Elevated levels of chemokines have been found in the synovial fluid of rheumatoid arthritis patients, chronic and acute rejecting tissue from transplant patients and in the nasal secretions of allergic rhinitis patients following allergen exposure (Teran, et al., J. Immunol., 1806-1812 (1996), and Kuna et al., J. Allergy Clin. Immunol. 321 (1994)). Antibodies which interfere with the chemokine/receptor interaction by neutralizing MIP-1α or gene disruption have provided direct evidence for the role of MIP-1α and RANTES in disease by limiting the recruitment of monocytes and CD8+ lymphocytes (Smith et al., J. Immunol, 153, 4704 (1994) and Cook et al., Science, 269, 1583 (1995)). Together this data demonstrates that CCR1 receptor antagonists would potentially be an effective treatment of several immune based diseases. The compounds described within are potent and selective antagonists of the CCR1 receptor.

### SUMMARY OF THE INVENTION

The present invention relates to a compound of the formula

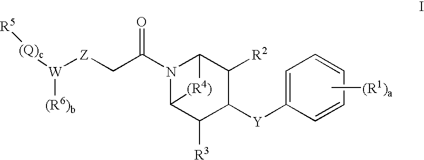

or pharmaceutically acceptable salts, tautomers, and pro-drugs thereof; wherein

a is 1, 2, 3, 4 or 5;

b is 0, 1, 2, 3, or 4;

c is 0 or 1;

Q is (C₁-C₆)alkyl;

W is (C₆-C₁₀)aryl or (C₂-C₉)heteroaryl;

Y is oxygen, or NR⁸ wherein R⁸ is hydrogen or (C₁-C₆)alkyl;

Z is oxygen or NR⁹, where R⁹ is hydrogen, (C₁-C₆)alkyl, or acetyl;

each R¹ is independently selected from the group consisting of: hydrogen, halo, cyano, nitro, trifluoromethyl, trifluoromethoxy, (C₁-C₆)alkyl, hydroxy or (C₁-C₆)alkylcarbonyloxy, (C₁-C₆)alkoxy;

R² and R³ are each independently hydrogen-or (C₁-C₆)alkyl optionally substituted with 1 to 3 halo groups;

R⁴ is (C₁-C₆)alkylene or —(CH₂)ₓ—O—(CH₂)_{y}—, wherein x and y are each independently 1 or 2;

R⁵ is selected from a list consisting of hydrogen, halo, (C₁-C₆)alkyl optionally substituted with 1 to 3 halo groups, [(C₁-C₆)alkyl]₂amino(C₁-C₆)alkylaminocarbonyl, amino(C₁-C₆)alkylaminocarbonyl, (C₁-C₆)alkylamino(C₁-C₆)alkylaminocarbonyl cyano, nitro, (C₁-C₆)alkoxy, aminocarbonyl, (C₁-C₆)alkylaminocarbonyl, [(C₁-C₆)alkyl]₂aminocarbonyl, (C₁-C₆)alkylsulfonylamino, (C₁-C₆)alkylsulfonylaminocarbonyl, ureido, aminosulfonyl, [(C₁-C₆)alkyl]₂aminosulfonyl, (C₁-C₆)alkylaminosulfonyl, [(C₁-C₆)alkyl]₂aminocarbonyl(C₁-C₆)alkylaminocarbonyl, (C₁-C₆)alkylaminocarbonyl(C₁-C₆)alkylaminocarbonyl, aminocarbonyl(C₁-C₆)alkylaminocarbonyl, (C₁-C₆)alkylsulfonylamino, hydroxy(C₁-C₆)alkylcarbonylamino, ureido(C₁-C₆)alkylaminocarbonyl, [(C₁-C₆)alkyl]₂ureido(C₁-C₆)alkylaminocarbonyl, (C₁-C₆)alkylureido(C₁-C₆)alkylaminocarbonyl, (C₂-C₉)heteroarylaminocarbonyl, carboxy, (C₁-C₆)alkoxy(C₁-C₆)alkyl(C₂-C₉)heterocyclecarbonyl, (C₂-C₉)heterocyclecarbonyl, hydroxy(C₂-C₉)heterocyclecarbonyl, aminocarbonyl(C₂-C₉)heterocyclecarbonyl, carboxy(C₂-C₉)heterocyclecarbonyl, amino(C₂-C₉)heteroaryl(C₁-C₆)alkyl, (C₁-C₆)alkylamino(C₂′-C₉)heteroaryl(C₁-C₆)alkyl, [(C₁-C₆)alkyl]₂amino(C₂-C₉)heteroaryl(C₁-C₆)alkyl, (C₂-C₉)heteroarylamino(C₁-C₆)alkyl, (C₂-C₉)heteroarylaminocarbonyl(C₁-C₆)alkoxy, (C₁-C₆)alkylsulfonylaminocarbonyl(C₁-C₆)alkoxy, aminocarbonyl(C₁-C₆)alkoxy, carboxy(C₁-C₆)alkoxy, carboxy(C₁-C₆)alkylcarbonylthiol, hydroxysulfonyl(C₁-C₆)alkylthiol, aminosulfonyl, (C₁-C₆)alkylcarbonylaminosulfonyl, hydroxy(C₁-C₆)alkylcarbonylaminosulfonyl, (C₁-C₆)alkoxycarbonylaminosulfonyl, (C₁-C₆)alkoxy(C₁-C₆)alkylcarbonylaminosulfonyl, hydroxysulfonyl, hydroxy, hydroxy(C₁-C₆)alkylaminocarbonyl, carboxy(C₂-C₉)heterocycloxy or [carboxy][amino](C₁-C₆)alkoxy, aminocarbonyl(C₁-C₆)alkylcarbonylamino, (C₁-C₆)alkylaminocarbonyl(C₁-C₆)alkylcarbonylamino, [(C₁-C₆)alkyl]₂aminocarbonyl(C₁-C₆)alkylcarbonylamino, amino(C₁-C₆)alkylcarbonylamino, (C₁-C₆)alkylamino(C₁-C₆)alkylcarbonylamino, [(C₁-C₆)alkyl]₂amino(C₁-C₆)alkylcarbonylamino, ureido(C₁-C₆)alkylcarbonylamino, (C₁-C₆)alkylureido(C₁-C₆)alkylcarbonylamino, [(C₁-C₆)alkyl]₂ureido(C₁-C₆)alkylcarbonylamino, amino(C₁-C₆)alkylsulfonylamino, amino(C₁-C₆)alkylcarbonylaminosulfonyl, (C₁-C₆)alkylamino(C₁-C₆)alkylcarbonylaminosulfonyl, [(C₁-C₆)alkyl]₂amino(C₁-C₆)alkylcarbonylaminosulfonyl, aminosulfonylamino, (C₁-C₆)alkylaminosulfonylamino, [(C₁-C₆)alkyl]₂aminosulfonylamino, (C₂-C₉)heterocycloxy, (C₂-C₉)heteroaryloxy, (C₂-C₉)heterocycleamino, (C₂-C₉)heteroarylamino, amino, (C₁-C₆)alkylamino, [(C₁-C₆)alkyl]₂amino, amino(C₁-C₆)alkoxy, (C₁-C₆)alkylamino(C₁-C₆)alkoxy, [(C₁-C₆)alkyl]₂amino(C₁-C₆)alkoxy, amino(C₁-C₆)alkylamino, (C₁-C₆)alkylcarbonylamino(C₁-C₆)alkylamino, ureido(C₁-C₆)alkylamino, hydroxy(C₁-C₆)alkylamino, (C₁-C₆)alkoxy(C₁-C₆)alkylamino, and (C₁-C₆)alkylsulfonylamino(C₁-C₆)alkylamino.

each R⁶ is independently selected from a list consisting of: hydrogen, halo, (C₁-C₆)alkyl optionally substituted with 1 to 3 halo groups; cyano, (C₁-C₆)alkoxy, aminocarbonyl, carboxy, (C₁-C₆)alkylcarbonyl, nitro, or (C₁-C₆)alkoxy optionally substituted by 1 to 3 halo groups.

Preferred compounds of the formula I include those wherein R¹ is halo, and a is 1 or 2.

Preferred compounds of the formula I include those wherein Y is oxygen.

Preferred compounds of the formula I include those wherein Z is oxygen.

Preferred compounds of the formula I include those wherein Z is NH.

Preferred compounds of the formula I include those wherein R⁴ is a —CH₂—CH₂— diradical.

Preferred compounds of the formula I include those wherein R⁴ is ‘cis’ to the Y group and R² and R³ are each hydrogen.

Preferred compounds of the formula I include those wherein W is phenyl.

Preferred compounds of the formula I include those d wherein W is pyridyl.

Preferred compounds of the formula I include those wherein c is 0, and R⁵ is selected from the group consisting of aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy.

Preferred compounds of the formula I include those wherein c is 1, and R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy.

Preferred compounds of the formula I include those wherein b is 0, 1 or 2, and R⁶ is selected from the group consisting of halo, (C₁-C₆)alkyl, cyano, or (C₁-C₆)alkylcarbonyl.

Preferred compounds of the formula I include those wherein R¹ is halo; a is 1 or 2; Y is oxygen; Z is oxygen; R⁴ is a —CH₂—CH₂— diradical; R⁴ is ‘cis’ to the Y group and R² and R³ are each hydrogen; W is phenyl; b is 0, 1 or 2; c is 0; R⁵ is selected from the group consisting of aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy; and R⁶ is selected from the group consisting of halo, (C₁-C₆)alkyl, cyano, or (C₁-C₆)alkylcarbonyl.

Preferred compounds of the formula I include those wherein R¹ is halo; a is 1 or 2; Y is oxygen; Z is oxygen or NH; R⁴ is a —CH₂—CH₂— diradical; R⁴ is ‘cis’ to the Y group and R² and R³ are each hydrogen; W is pyridyl; b is 0, 1 or 2; c is 0; R⁵ is selected from the group consisting of: aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy; and R⁶ is selected from the group consisting of halo, (C₁-C₆)alkyl, cyano, or (C₁-C₆)alkylcarbonyl.

Preferred compounds of the formula I include those wherein R₁ is halo; a is 1 or 2; Y is oxygen; Z is oxygen; R⁴ is a —CH₂—CH₂— diradical; R⁴ is ‘cis’ to the Y group and R² and R³ are each hydrogen; W is phenyl; b is 0, 1 or 2; c is 1; R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy; and R⁶ is selected from the group consisting of halo, (C₁-C₆)alkyl, cyano, or (C₁-C₆)alkylcarbonyl.

Preferred compounds of the formula I include those wherein R¹ is halo; a is 1 or 2; Y is oxygen; Z is oxygen or NH; R⁴ is a —CH₂—CH₂— diradical; R⁴ is ‘cis’ to the Y group and R² and R³ are each hydrogen; W is pyridyl; b is 0, 1 or 2; c is 1; R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy; and R⁶ is selected from the group consisting of halo, (C₁-C₆)alkyl, cyano, or (C₁-C₆)alkylcarbonyl.

The most preferred compounds of the formula I include those selected from the group consisting of:

5-Chloro-2-{2-[(trans)-3-(4-fluoro-phenoxy)-8-aza-bicyclo[3.2.1]oct-8-yl]-2-oxo-ethoxy}-benzamide;

5-Chloro-2-{2-[(cis)-3-(4-fluoro-phenoxy)-8-aza-bicyclo[3.2.1]oct-8-yl]-2-oxo-ethoxy}-benzamide;

2-{2-[(cis)-3-(4-Fluoro-phenoxy)-8-aza-bicyclo[3.2.1]oct-8-yl]-2-oxo-ethoxy}-4-methoxy-benzamide;

5-Chloro-2-{2-[(cis)-3-(4-fluoro-phenoxy)-8-aza-bicyclo[3.2.1]oct-8-yl]-2-oxo-ethoxy}-benzenesulfonamide;

N-Carbamoylmethyl-5-chloro-2-{2-[(cis)-3-(4-fluoro-phenoxy)-8-aza-bicyclo[3.2.1]oct-8-yl]-2-oxo-ethoxy}-benzamide;

(5-Chloro-2-{2-[(cis)-3-(4-fluoro-phenoxy)-8-aza-bicyclo[3.2.1]oct-8-yl]-2-oxo-ethoxy}-benzoylamino)-acetic acid;

N-(5-Chloro-2-{2-[(cis)-3-(4-fluoro-phenoxy)-8-aza-bicyclo[3.2.1]oct-8-yl]-2-oxo-ethoxy}-phenyl)-methanesulfonamide;

N-(5-Chloro-2-{2-[(cis)-3-(4-fluoro-phenoxy)-8-aza-bicyclo[3.2.1]oct-8-yl]-2-oxo-ethoxy}-phenyl)-3-hydroxy-3-methyl-butyramide;

(5-Chloro-2-{2-[(cis)-3-(4-fluoro-phenoxy)-8-aza-bicyclo[3.2.1]oct-8-yl]-2-oxo-ethoxy}-phenyl)-urea;

(5-Chloro-2-{2-[(trans)-3-(4-fluoro-phenoxy)-8-aza-bicyclo[3.2.1]oct-8-yl]-2-oxo-ethoxy}-phenyl)-urea;

5-Chloro-2-{2-[(cis)-3-(4-fluoro-phenoxy)-8-aza-bicyclo[3.2.1]oct-8-yl]-2-oxo-ethoxy}-N-(2-ureido-ethyl)-benzamide;

5-Chloro-2-{2-[(cis)-3-(4-fluoro-phenoxy)-8-aza-bicyclo[3.2.1]oct-8-yl]-2-oxo-ethoxy}-N-(2H-tetrazol-5-yl)-benzamide;

5-Chloro-2-{2-[(cis)-3-(4-fluoro-phenoxy)-8-aza-bicyclo[3.2.1]oct-8-yl]-2-oxo-ethoxy}-benzoic acid;

5-Chloro-2-{2-[(cis)-3-(4-fluoro-phenoxy)-8-aza-bicyclo[3.2.1]oct-8-yl]-2-oxo-ethoxy}-N-pyridin-2-yl-benzamide;

2-[4-Chloro-2-((2R)-2-methoxymethyl-pyrrolidine-1-carbonyl)-phenoxy]-1-[(cis)-3-(4-fluoro-phenoxy)-8-aza-bicyclo[3.2.1]oct-8-yl]-ethanone;

2-[4-Chloro-2-(morpholine-4-carbonyl)-phenoxy]-1-(cis)-[3-(4-fluoro-phenoxy)-8-aza-bicyclo[3.2.1]oct-8-yl]-ethanone;

N-(2-{2-[3-(4-Fluoro-phenoxy)-8-aza-bicyclo[3.2.1]oct-8-yl]-2-oxo-ethoxy}-5-trifluoromethyl-phenyl)-methanesulfonamide;

5-Chloro-N-(2-dimethylamino-ethyl)-2-{2-[(cis)-3-(4-fluoro-phenoxy)-8-aza-bicyclo[3.2.1]oct-8-yl]-2-oxo-ethoxy}-benzamide;

2-[4-Chloro-2-((3S)-3-hydroxy-pyrrolidine-1-carbonyl)-phenoxy]-1-[(cis)-3-(4-fluoro-phenoxy)-8-aza-bicyclo[3.2.1]oct-8-yl]-ethanone;

2-[4-Chloro-2-((2S)-2-methoxymethyl-pyrrolidine-1-carbonyl)-phenoxy]-1-[(cis)-3-(4-fluoro-phenoxy)-8-aza-bicyclo[3.2.1]oct-8-yl]-ethanone;

2-[4-Chloro-2-((3R)-3-hydroxy-pyrrolidine-1-carbonyl)-phenoxy]-1-[(cis)-3-(4-fluoro-phenoxy)-8-aza-bicyclo[3.2.1]oct-8-yl]-ethanone;

1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2S)-2-carboxylic acid;

N-(2-Amino-ethyl)-5-chloro-2-{2-[3-(4-fluoro-phenoxy)-8-aza-bicyclo[3.2.1]oct-8-yl]-2-oxo-ethoxy}-benzamide;

1-(5-Chloro-2-{2-[(cis)-3-(4-fluoro-phenoxy)-8-aza-bicyclo[3.2.1]oct-8-yl]-2-oxo-ethoxy}-benzoyl)-(4S)-4-hydroxy-pyrrolidine-(2S)-2-carboxylic acid amide;

1-(5-Chloro-2-{2-[(cis)-3-(4-fluoro-phenoxy)-8-aza-bicyclo[3.2.1]oct-8-yl]-2-oxo-ethoxy}-benzoyl)-(4S)-4-hydroxy-pyrrolidine-(2S)-2-carboxylic acid;

1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2S)-2-carboxylic acid amide;

1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy pyrrolidine-(2R)-2-carboxylic acid amide;

1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2R)-2-carboxylic acid;

2-(5-Chloro-quinolin-8-yloxy)-1-[(cis)-3-(4-fluoro-phenoxy)-8-aza-bicyclo[3.2.1]oct-8-yl]-ethanone;

(5-Chloro-2-{2-[(cis)-3-(4-fluoro-phenoxy)-8-aza-bicyclo[3.2.1]oct-8-yl]-2-oxo-ethoxy}-phenyl)-acetic acid;

5-Chloro-2-{2-[(trans)-7-(4-fluoro-phenoxy)-3-oxa-9-aza-bicyclo[3.3.1]non-9-yl]-2-oxo-ethoxy}-benzamide;

2-(5-Chloro-2-{2-[(cis)-3-(4-fluoro-phenoxy)-8-aza-bicyclo[3.2.1]oct-8-yl]-2-oxo-ethoxy}-phenyl)-acetamide;

N-(5-Chloro-2-{2-[(cis)-3-(4-fluoro-phenoxy)-8-aza-bicyclo[3.2.1]oct-8-yl]-2-oxo-ethoxy}-benzoyl)-methanesulfonamide;

N-[(5-Chloro-2-{2-[(cis)-3-(4-fluoro-phenoxy)-8-aza-bicyclo[3.2.1]oct-8-yl]-2-oxo-ethoxy}-phenyl)-acetyl]-methanesulfonamide;

2-[2-(5-Amino-tetrazol-1-ylmethyl)-4-chloro-phenoxy]-1-[(cis)-3-(4-fluoro-phenoxy)-8-aza-bicyclo[3.2.1]oct-8-yl]-ethanone;

2-[2-(5-Amino-tetrazol-2-ylmethyl)-4-chloro-phenoxy]-1-[(cis)-3-(4-fluoro-phenoxy)-8-aza-bicyclo[3.2.1]oct-8-yl]-ethanone;

5-Chloro-2-{2-[(cis)-3-(4-fluoro-phenoxy)-8-aza-bicyclo[3.2.1]oct-8-yl]-2-oxo-ethoxy}-N-pyrimidin-4-yl-benzamide;

2-[4-Chloro-2-(1H-tetrazol-5-yl)-phenoxy]-1-[(cis)-3-(4-fluoro-phenoxy)-8-aza-bicyclo[3.2.1]oct-8-yl]-ethanone;

2-[4-Chloro-2-(1H-tetrazol-5-ylmethyl)-phenoxy]-1-[(cis)-3-(4-fluoro-phenoxy)-8-aza-bicyclo[3.2.1]oct-8-yl]-ethanone;

(5-Chloro-2-{2-[(trans)-3-(4-fluoro-phenoxy)-8-aza-bicyclo[3.2.1]oct-8-yl]-2-oxo-ethoxy}-phenyl)-acetic acid;

N-[(5-Chloro-2-{2-[(trans)-3-(4-fluoro-phenoxy)-8-aza-bicyclo[3.2.1]oct-8-yl]-2-oxo-ethoxy}-phenyl)-acetyl]-methanesulfonamide;

2-(5-Chloro-2-{2-[(trans)-3-(4-fluoro-phenoxy)-8-aza-bicyclo[3.2.1]oct-8-yl]-2-oxo-ethoxy}-phenyl)-acetamide;

2-{4-Chloro-2-[(1H-tetrazol-5-ylamino)-methyl]-phenoxy}-1-[(cis)-3-(4-fluoro-phenoxy)-8-aza-bicyclo[3.2.1]oct-8-yl]-ethanone;

(5-Chloro-2-{2-[(trans)-7-(4-fluoro-phenoxy)-3-oxa-9-aza-bicyclo[3.3.1]non-9-yl]-2-oxo-ethoxy}-phenyl)-acetic acid;

2-[4-Chloro-2-(1-hydroxy-1-methyl-ethyl)-phenoxy]-1-(cis)-[3-(4-fluoro-phenoxy)-8-aza-bicyclo[3.2.1]oct-8-yl]-ethanone;

N-[(5-Chloro-2-{2-[(trans)-7-(4-fluoro-phenoxy)-3-oxa-9-aza-bicyclo[3.3.1]non-9-yl]-2-oxo-ethoxy}-phenyl)-acetyl]-methanesulfonamide;

(5-Chloro-2-{2-[(cis)-3-(4-fluoro-phenoxy)-8-aza-bicyclo[3.2.1]oct-8-yl]-2-oxo-ethoxy}-benzyloxy)-acetic acid;

2-(5-Chloro-2-{2-[(cis)-3-(4-fluoro-phenoxy)-8-aza-bicyclo[3.2.1]oct-8-yl]-2-oxo-ethoxy}-benzyloxy)-N-(1H-tetrazol-5-yl)-acetamide;

N-[(5-Chloro-2-{2-[(cis)-3-(4-fluoro-phenoxy)-8-aza-bicyclo[3.2.1]oct-8-yl]-2-oxo-ethoxy}-benzyloxy)-acetyl]-methanesulfonamide;

2-(5-Chloro-2-{2-[(cis)-3-(4-fluoro-phenoxy)-8-aza-bicyclo[3.2.1]oct-8-yl]-2-oxo-ethoxy}-benzyloxy)-acetamide;

(5-Bromo-2-{2-[(cis)-3-(4-fluoro-phenoxy)-8-aza-bicyclo[3.2.1]oct-8-yl]-2-oxo-ethoxy}phenyl)-acetic acid;

2-(5-Bromo-2-{2-[(cis)-3-(4-fluoro-phenoxy)-8-aza-bicyclo[3.2.1]oct-8-yl]-2-oxo-ethoxy}-phenyl)-acetamide;

N-[(5-Bromo-2-{2-[(cis)-3-(4-fluoro-phenoxy)-8-aza-bicyclo[3.2.1]oct-8-yl]-2-oxo-ethoxy}-phenyl)-acetyl]-methanesulfonamide;

(5-Chloro-2-{2-[(cis)-3-(4-fluoro-phenoxy)-8-aza-bicyclo[3.2.1]oct-8-yl]-2-oxo-ethoxy}-phenyl)-methanesulfonamide;

N-Acetyl-C-(5-chloro-2-{2-[(cis)-3-(4-fluoro-phenoxy)-8-aza-bicyclo[3.2.1]oct-8-yl]-2-oxo-ethoxy}-phenyl)-methanesulfonamide;

(5-Bromo-2-{2-[(cis)-3-(4-fluoro-phenoxy)-8-aza-bicyclo[3.2.1]oct-8-yl]-2-oxo-ethoxy}-phenyl)-methanesulfonamide;

N-Acetyl-C-(5-bromo-2-{2-[(cis)-3-(4-fluoro-phenoxy)-8-aza-bicyclo[3.2.1]oct-8-yl]-2-oxo-ethoxy}-phenyl)-methanesulfonamide;

C-(5-Chloro-2-{2-[(cis)-3-(4-fluoro-phenoxy)-8-aza-bicyclo[3.2.1]oct-8-yl]-2-oxo-ethoxy}-phenyl)-N-(2-hydroxy-2-methyl-propionyl)-methanesulfonamide;

C-(5-Chloro-2-{2-[(cis)-3-(4-fluoro-phenoxy)-8-aza-bicyclo[3.2.1]oct-8-yl]-2-oxo-ethoxy}-phenyl)-N-hydroxyacetyl-methanesulfonamide;

C-(5-Chloro-2-{2-[(cis)-3-(4-fluoro-phenoxy)-8-aza-bicyclo[3.2.1]oct-8-yl]-2-oxo-ethoxy}-phenyl)-N-(methoxycarbonyl)-methanesulfonamide;

3-(5-Chloro-2-{2-[(cis)-3-(4-fluoro-phenoxy)-8-aza-bicyclo[3.2.1]oct-8-yl]-2-oxo-ethoxy}-phenyl)-propionic acid;

C-(5-Chloro-2-{2-[(cis)-3-(4-fluoro-phenoxy)-8-aza-bicyclo[3.2.1]oct-8-yl]-2-oxo-ethoxy}-phenyl)-N-(1-hydroxy-cyclopropanecarbonyl)-methanesulfonamide;

N-[3-(5-Chloro-2-{2-[(cis)-3-(4-fluoro-phenoxy)-8-aza-bicyclo[3.2.1]oct-8-yl]-2-oxo-ethoxy}-phenyl)-propionyl]-methanesulfonamide;

C-(5-Chloro-2-{2-[(cis)-3-(4-fluoro-phenoxy)-8-aza-bicyclo[3.2.1]oct-8-yl]-2-oxo-ethoxy}-phenyl)-N-methoxyacetyl-methanesulfonamide;

4-{2-[(cis)-3-(4-Fluoro-phenoxy)-8-aza-bicyclo[3.2.1]oct-8-yl]-2-oxo-ethoxy}-benzoic acid;

1-[(cis)-3-(4-Fluoro-phenoxy)-8-aza-bicyclo[3.2.1]oct-8-yl]-2-phenoxy-ethanone;

2-(4-Bromo-phenoxy)-1-[(cis)-3-(4-fluoro-phenoxy)-8-aza-bicyclo[3.2.1]oct-8-yl]-ethanone;

1-[(cis)-3-(4-Fluoro-phenoxy)-8-aza-bicyclo[3.2.1]oct-8-yl]-2-(4-trifluoromethyl-phenoxy)-ethanone;

1-[(cis)-3-(4-Fluoro-phenoxy)-8-aza-bicyclo[3.2.1]oct-8-yl]-2-p-tolyloxy-ethanone;

2-(4-Chloro-phenoxy)-1-(cis)-[3-(4-fluoro-phenoxy)-8-aza-bicyclo[3.2.1]oct-8-yl]-ethanone;

(5-Chloro-2-{2-[(cis)-3-(4-fluoro-phenoxy)-8′-aza-bicyclo[3.2.1]oct-8-yl]-2-oxo-ethoxy}-phenyl)-methanesulfonic acid;

2-(2-Acetyl-4-chloro-phenoxy)-1-[(cis)-3-(4-fluoro-phenoxy)-8-aza-bicyclo[3.2.1]oct-8-yl]-ethanone;

5-Chloro-2-{2-[(cis)-3-(4-fluoro-phenoxy)-8-aza-bicyclo[3.2.1]oct-8-yl]-2-oxo-ethoxy}-N-methyl-benzamide;

5-Bromo-2-{2-[(cis)-3-(4-fluoro-phenoxy)-8-aza-bicyclo[3.2.1]oct-8-yl]-2-oxo-ethoxy}-benzamide;

2-(4-Chloro-2-hydroxymethyl-phenoxy)-1-[(cis)-3-(4-fluoro-phenoxy)-8-aza-bicyclo[3.2.1]oct-8-yl]-ethanone;

2-(4-Bromo-2-hydroxymethyl-phenoxy)-1-(cis)-[3-(4-fluoro-phenoxy)-8-aza-bicyclo[3.2.1]oct-8-yl]-ethanone;

2-(4-Chloro-2-hydroxy-phenoxy)-1-[(cis)-3-(4-fluoro-phenoxy)-8-aza-bicyclo[3.2.1]oct-8-yl]-ethanone;

(5-Chloro-2-{2-[(cis)-3-(4-fluoro-phenoxy)-8-aza-bicyclo[3.2.1]oct-8-yl]-2-oxo-ethoxy}-phenoxy)-acetic acid;

2-(4-Bromo-2-hydroxy-phenoxy)-1-[(cis)-3-(4-fluoro-phenoxy)-8-aza-bicyclo[3.2.1]oct-8-yl]-ethanone;

5-Chloro-2-{(2-[(cis)-3-(4-fluoro-phenoxy)-8-aza-bicyclo[3.2.1]oct,-8-yl]-2-oxo-ethoxy}-N-(2-hydroxy-ethyl)-benzamide;

5-Chloro-2-{2-[(cis)-3-(4-fluoro-phenoxy)-8-aza-bicyclo[3.2.1]oct-8-yl]-2-oxo-ethoxy}-N-(3-hydroxy-propyl)-benzamide;

4-(5-Chloro-2-{2-[(cis)-3-(4-fluoro-phenoxy)-8-aza-bicyclo[3.2.1]oct-8-yl]-2-oxo-ethoxy}-phenoxy)-pyrrolidine-(2S)-2-carboxylic acid;

(2S)-2-Amino-4-(5-chloro-2-{2-[(cis)-3-(4-fluoro-phenoxy)-8-aza-bicyclo[3.2.1]oct-8-yl]-2-oxo-ethoxy}-phenoxy)-butyric acid;

(cis)-5-Chloro-2-{2-[3-(4-fluoro-phenoxy)-8-aza-bicyclo[3.2.1]oct-8-yl]-2-oxo-ethylamino}-nicotinic acid;

5-Chloro-2-{2-[3-(4-fluoro-phenoxy)-8-aza-bicyclo[3.2.1]oct-8-yl]-2-oxo-ethylamino}-nicotinamide;

(cis)-5-Chloro-N-(2-dimethylamino-ethyl)-2-{2-[3-(4-fluoro-phenoxy)-8-aza-bicyclo[3.2.1]oct-8-yl]-2-oxo-ethylamino}-nicotinamide;

(cis)-N-(2-Amino-ethyl)-5-chloro-2-{2-[3-(4-fluoro-phenoxy)-8-aza-bicyclo[3.2.1]oct-8-yl]-2-oxo-ethylamino}-nicotinamide;

[(cis)-(5-Chloro-2-{2-[3-(4-fluoro-phenoxy)-8-aza-bicyclo[3.2.1]oct-8-yl]-2-oxo-ethylamino}-pyridine-3-carbonyl)-amino]-acetic acid;

2-[5-Chloro-3-(morpholine-4-carbonyl)-pyridin-2-ylamino]-1-[(cis)-3-(4-fluoro-phenoxy)-8-aza-bicyclo[3.2.1]oct-8-yl]-ethanone;

2-[5-Chloro-3-((3S)-3-hydroxy-pyrrolidine-1-carbonyl)-pyridin-2-ylamino]-1-[(cis)-3-(4-fluoro-phenoxy)-8-aza-bicyclo[3.2.1]oct-8-yl]-ethanone;

2-[5-Chloro-3-((3R)-3-hydroxy-pyrrolidine-1-carbonyl)-pyridin-2-ylamino]-1-[(cis)-3-(4-fluoro-phenoxy)-8-aza-bicyclo[3.2.1]oct-8-yl]-ethanone;

2-[5-Chloro-3-((2S)-2-methoxymethyl-pyrrolidine-1-carbonyl)-pyridin-2-ylamino]-1-[(cis)-3-(4-fluoro-phenoxy)-8-aza-bicyclo[3.2.1]oct-8-yl]-ethanone;

2-[5-Chloro-3-((2R)-2-methoxymethyl-pyrrolidine-1-carbonyl)-pyridin-2-ylamino]-1-[(cis)-3-(4-fluoro-phenoxy)-8-aza-bicyclo[3.2.1]oct-8-yl]-ethanone;

(cis)-N-Carbamoylmethyl-5-chloro-2-{2-[3-(4-fluoro-phenoxy)-8-aza-bicyclo[3.2.1]oct-8-yl]-2-oxo-ethylamino}-nicotinamide;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2S)-2-carboxylic acid amide;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4S)-4-hydroxy-pyrrolidine-(2S)-2-carboxylic acid amide;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2R)-2-carboxylic acid amide;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2S)-2-carboxylic acid;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4S)-4-hydroxy-pyrrolidine-(2S)-2-carboxylic acid;

1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2R)-2-carboxylic acid;

5-Chloro-2-{2-[(cis)-3-(4-fluoro-phenoxy)-8-aza-bicyclo[3.2.1]oct-8-yl]-2-oxo-ethylamino}-N-pyrimidin-4-yl-nicotinamide;

N-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-methanesulfonamide;

5-Chloro-2-{2-[(cis)-3-(4-fluoro-phenoxy)-8-aza-bicyclo[3.2.1]oct-8-yl]-2-oxo-ethylamino}-N-pyridin-2-yl-nicotinamide;

5-Chloro-2-{2-[(cis)-3-(4-fluoro-phenoxy)-8-aza-bicyclo[3.2.1]oct-8-yl]-2-oxo-ethoxy}-nicotinamide;

2-(3-Amino-5-chloro-pyridin-2-yloxy)-1-[(cis)-3-(4-fluoro-phenoxy)-8-aza-bicyclo[3.2.1]oct-8-yl]-ethanone;

(5-Chloro-2-{2-[(cis)-3-(4-fluoro-phenoxy)-8-aza-bicyclo[3.2.1]oct-8-yl]-2-oxo-ethoxy}-pyridin-3-yl)-urea;

2-Amino-N-(5-chloro-2-{2-[(cis)-3-(4-fluoro-phenoxy)-8-aza-bicyclo[3.2.1]oct-8-yl]-2-oxo-ethoxy}-pyridin-3-yl)-acetamide;

N-(5-Chloro-2-{2-[(cis)-3-(4-fluoro-phenoxy)-8-aza-bicyclo[3.2.1]oct-8-yl]-2-oxo-ethoxy}-pyridin-3-yl)-succinamic acid; and

N-Acetyl-5-chloro-2-{2-[(cis)-3-(4-fluoro-phenoxy)-8-aza-bicyclo[3.2.1]oct-8-yl]-2-oxo-ethoxy}-nicotinamide.

The present invention also relates to a pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. Pharmaceutical compositions of this invention are also potentially useful for the treatment or prevention of cancer metastasis, including but not limited to breast cancer. Pharmaceutical compositions of this invention may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Pharmaceutical compositions of this invention may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, preferably a human, comprising an amount of a compound of the formula I or a pharmaceutically acceptable salt thereof effective in treating or preventing such a disorder or condition and a pharmaceutically acceptable carrier.

The present invention also relates to a pharmaceutical composition for treating or preventing a disorder or condition that can be treated or prevented by inhibiting chemokine binding to the receptor CCR1 in a mammal, preferably a human, comprising an amount of a compound of the formula I, or a pharmaceutically acceptable salt thereof, effective in treating or preventing such disorder or condition and a pharmaceutically acceptable carrier. Examples of such disorders and conditions are those enumerated in the preceding paragraph.

The present invention also relates to a method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, preferably a human, comprising administering to a mammal in need of such treatment or prevention an amount of a compound of the formula I, or a pharmaceutically acceptable salt thereof, that is effective in treating or preventing such disorder or condition.

The present invention also relates to a method for treating or preventing a disorder or condition that can be treated or prevented by antagonizing the CCR1 receptor in a mammal, preferably a human, comprising administering to a mammal in need of such treatment or prevention an amount of a compound of the formula I, or a pharmaceutically acceptable salt thereof, that is effective in treating or preventing such disorder or condition.

The present invention also relates to a pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. Pharmaceutical compositions of this invention are also potentially useful for the treatment or prevention of cancer metastasis, including but not limited to breast cancer. Pharmaceutical compositions of this invention may also inhibit the production of metalloproteinases and cytokines at inflammatory sites (including but not limited to MMP9, TNF, IL-1, and 1L-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Pharmaceutical compositions of this invention may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, preferably a human, comprising a CCR1 receptor antagonizing effective amount of a compound of the formula I, or a pharmaceutically acceptable salt thereof, and a pharmaceutically acceptable carrier.

The present invention also relates to a pharmaceutical composition for treating or preventing a disorder or condition that can be treated or prevented by antagonizing the CCR1 receptor in a mammal, preferably a human, comprising a CCR1 receptor antagonizing effective amount of a compound of the formula I, or a pharmaceutically acceptable salt thereof, and a pharmaceutically acceptable carrier.

The present invention also relates to a method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, preferably a human, comprising administering to a mammal in need of such treatment or prevention a CCR1 receptor antagonizing effective amount of a compound of formula I, or a pharmaceutically acceptable salt thereof.

### DETAILED DESCRIPTION OF THE INVENTION

### Preparation A

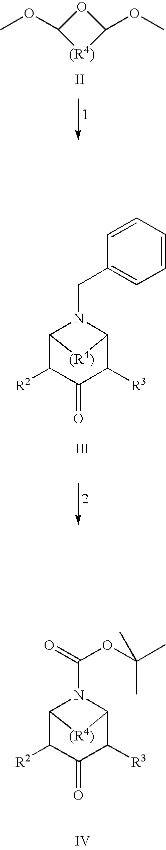

### Preparation B

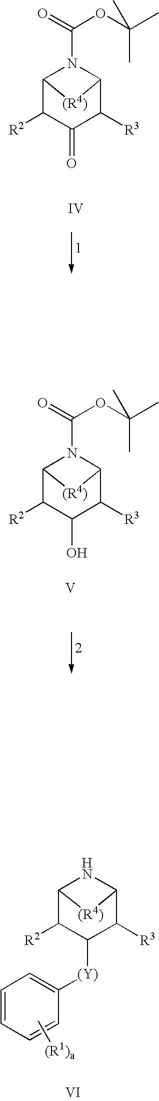

### Preparation C

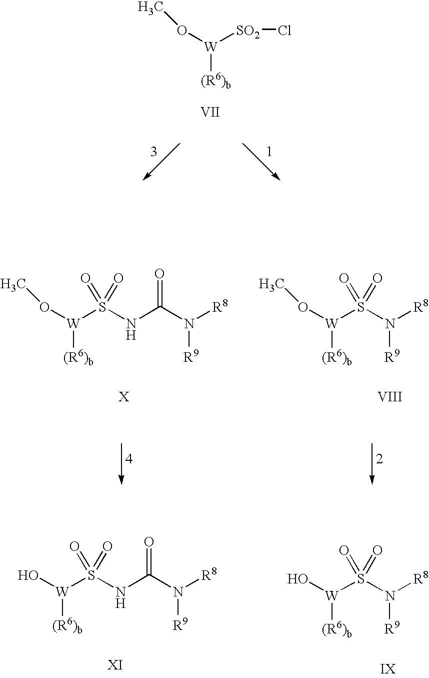

### Preparation D

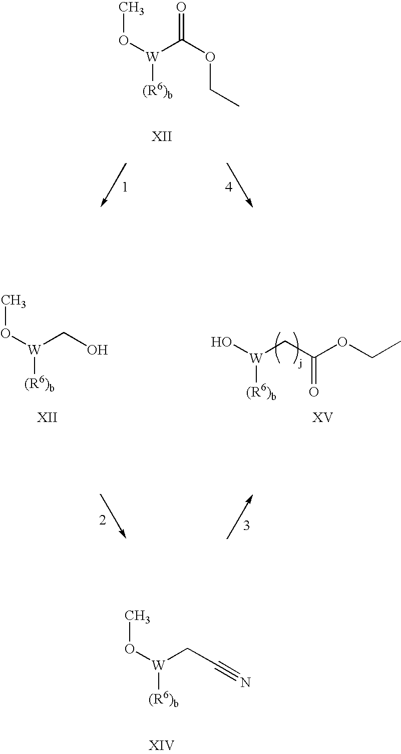

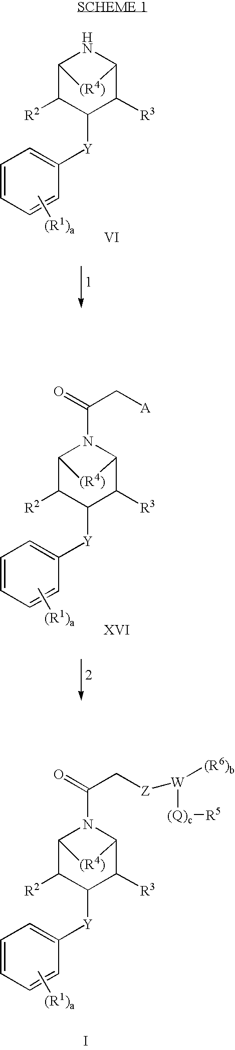

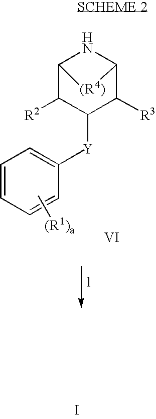

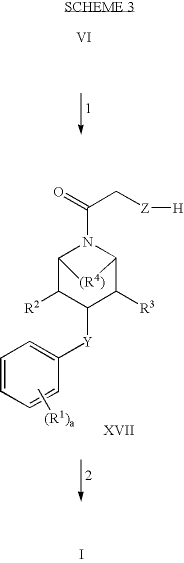

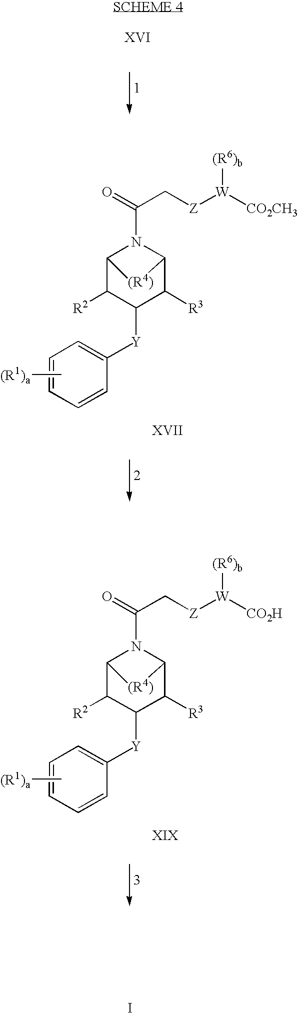

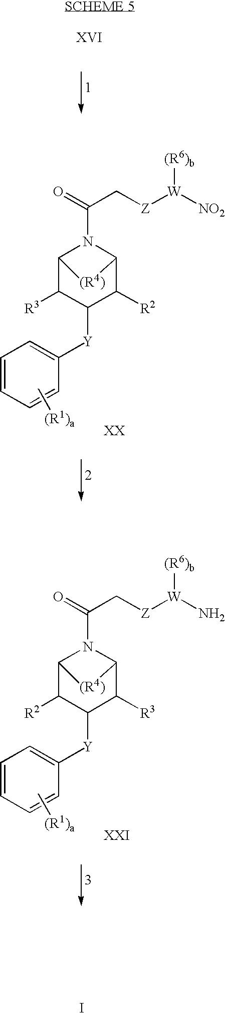

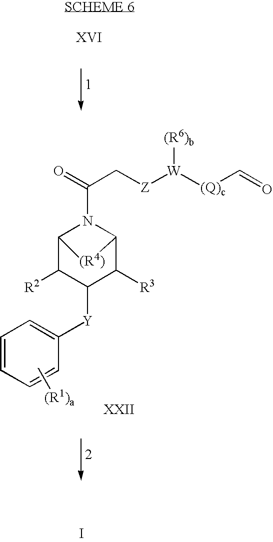

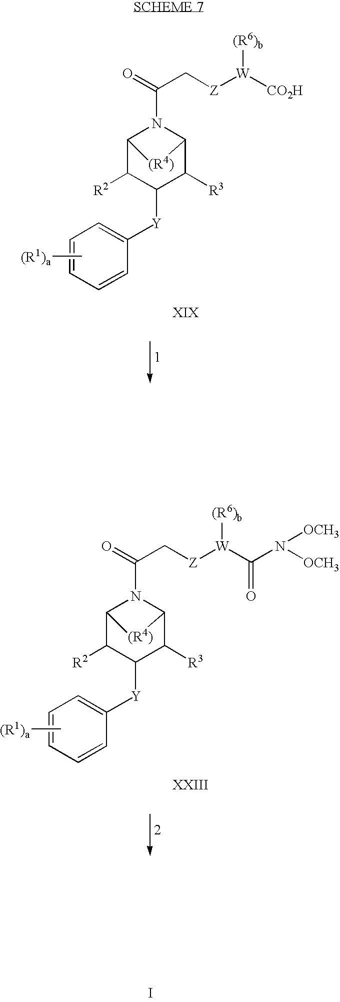

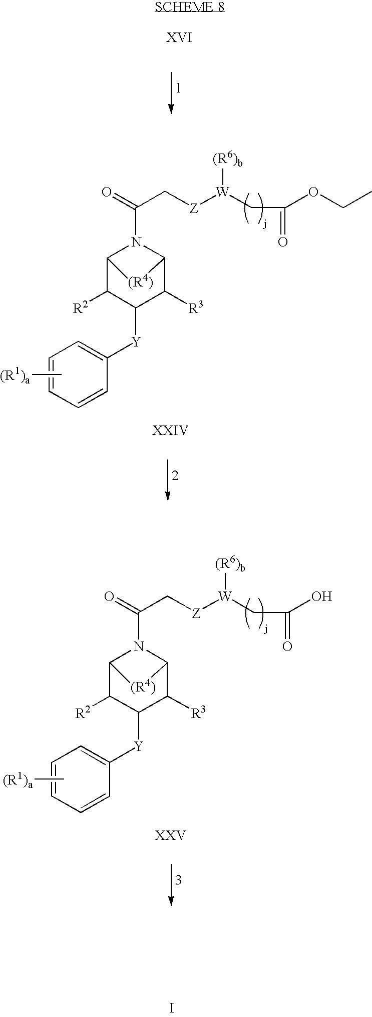

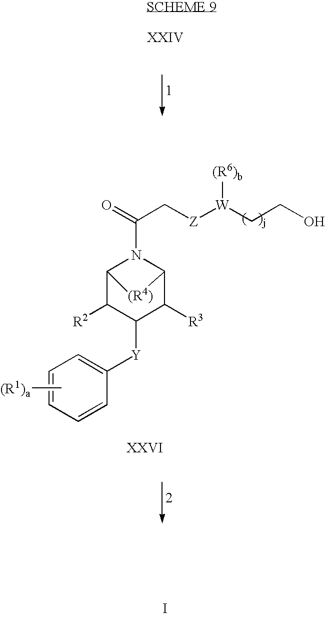

In reaction 1 of Preparation A, the compound of formula II, wherein R⁴ is (C₁-C₆)alkylene or —(CH₂)ₓ—O—(CH₂)_{y}—, wherein x and y are each independently 1 or 2, is converted to the corresponding compound of formula III by reacting with an amine, such as benzyl amine, and a compound of the formula:

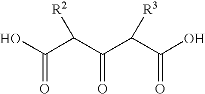

wherein R² and R³ are each independently hydrogen or (C₁-C₆)alkyl, in the presence of an acid, such as 0.25 M aqueous hydrochloric acid. The reaction is stirred at ambient temperature for a period of time between about 30 minutes to about 2 hours, preferably about 1.5 hours, and then heated to a temperature between about 40° C. to about 60° C., preferably about 50° C., for a period of time between about 1 hour and about 4 hours, preferably about 2 hours.

In reaction 2 of Preparation A, the compound of formula III is converted to the corresponding compound of formula IV by shaking a solution of III in ethanol under a positive pressure of hydrogen gas in the presence of a catalyst, such as palladium hydroxide on carbon, and carbonic acid di-tert-butyl ester.

In reaction 1 of Preparation B, the compound of formula IV, which is either commercially available or has been prepared according to Preparation A, is converted to the corresponding compound of formula V by reacting with a reducing agent, such as L-selectride, in an aprotic solvent, such as tetrahydrofuran, to give a mixture of diastereomeric mixture of alcohols, which are separated at this stage by silica gel chromatography.

In reaction 2 of Preparation B the compound of formula V is then converted to the corresponding compound of formula VI by treating the alcohol so formed with triphenyl phosphine and diethyl azodicarboxylate in the presence of a nucleophile of the formula:

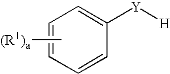

where in Y is oxygen and a is 1, 2, 3, 4 or 5. Finally, the resulting arylether is deprotected with trifluoro acetic acid in an aprotic solvent, such as methylene chloride, to give the corresponding compound of formula VI. In the case that Y is NH, a compound of formula IV is treated with a compound of the formula:

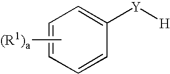

wherein Y is NH and a is 1, 2, 3, 4, or 5, in the presence of a reducing agent, such as sodium cyanoborohydride, in the presence of a polar aprotic solvent, such as dichloroethane. Deprotection with trifluoroacetic acid gives the corresponding compound of formula VI.

In reaction 1 of the Preparation C, the compound of formula VII is converted to the corresponding compound of formula VIII by reacting VII with an appropriate amine of the formula, HNR⁸R⁹, wherein R⁸ and R⁹ are each independently selected from a group, including but not limited to, hydrogen, a nitrogen containing (C₂-C₉)heterocycloalkyl or (C₂-C₉)heteroaryl group, or an optionally substituted (C₁-C₆)alkyl, or R¹⁸ and R¹⁹ are taken together with the nitrogen to which they are attached to form (C₂-C₉)heterocycloalkyl or (C₂-C₉)heteroaryl group, in the presence of a polar aprotic solvent, such as methylene chloride. The reaction mixture is stirred, at ambient temperature, for a time period between about 1 hour to about 24 hours, preferably about 12 hours.

In reaction 2 of Preparation C, the compound of formula VIII is converted to the corresponding compound of formula IX by reacting VIII with thiophenol in the presence of a base, such as sodium hydride, and a polar aprotic solvent, such as dimethylformamide. The reaction is heated to reflux for a time period between about 1 hour to about 10 hours, preferably about 4 hours.

In reaction 3 of Preparation C, the compound of formula VII is converted to the corresponding compound of formula X by reacting VII with sodium cyanate in the presence of pyridine and a polar aprotic solvent, such as acetonitrile. The reaction is stirred, at ambient temperature, for a time period between about 2 hours to about 18 hours, preferably about 10 hours. An appropriate amine of the formula HNR⁸R⁹, wherein R⁸ and R⁹ are each independently selected from a group, including but not limited to, hydrogen, a nitrogen containing (C₂-C₉)heterocycloalkyl or (C₂-C₉)heteroaryl group, or an optionally substituted (C₁-C₆)alkyl, or R¹⁸ and R¹⁹ are taken together with the nitrogen to which they are attached to form (C₂-C₉)heterocycloalkyl or (C₂-C₉)heteroaryl group, is then added and the reaction mixture so formed is stirred, at ambient temperature, for a time period between about 2 hours to about 24 hours, preferably about 8 hours.

In reaction 4 of Preparation C, the compound of formula X is converted to the corresponding compound of formula XI according to the procedure described above in reaction 2 of Preparation C.

In reaction 1 of Preparation D the compound of formula XII is converted to the corresponding compound of the formula XIII by treating with a reducing agent, such as lithium aluminum hydride, in an aprotic solvent, such as tetrahydrofuran. The reaction mixture is heated to reflux for a time period between 1 hour and 6 hours, preferably about 2 hours.

In reaction 2 of Preparation D the compound of formula XIII is converted to the corresponding compound of the formula XIV by first treating with an activating agent such as sulfonyl chloride, in the presence of an aprotic solvent, such as chloroform. The reaction is heated to reflux, for a time period between about 1 hour to about 10 hours, preferably about 3 hours. The resulting alkyl chloride is then treated with a cyanide source, such as potassium cyanide, in the presence of an aprotic solvent, such as acetonitrile. The reaction mixture is stirred at ambient temperature for a time period between about 1 hour to about 10 hours, preferably about 3 hours.

In reaction 3 of Preparation D the compound of formula XIV is converted to the compound of formula XV, wherein j is 1, by first treating XIV with base, such as potassium hydroxide in water. The reaction mixture is heated to reflux for a time period between about 1 hour to about 10 hours, preferably about 6 hours. The resulting carboxylate is treated with acid, such as 47% aqueous hydrogen bromide to produce the deprotected phenol. The reaction mixture is heated to reflux for a time period between about 10 hours to about 30 hours, preferably about 24 hours. The deprotected phenol is finally converted to the corresponding compound of formula XV, wherein j is 1, by refluxing in ethanol in the presence of an acid, such as sulfuric acid, for a time period between about 8 hours to about 16 hours, preferably about 12 hours.

In reaction 4 of Preparation D the compound of formula XII is converted to the corresponding compound of formula XV, wherein j is 2 or 3, by first treating the ester with a reducing agent, such as diisobutylaluminum hydride, in the presence of an aprotic solvent, such as toluene. The resulting aldehyde is treated with a phosphonium ylide derived from the phosphonium salt of the formula

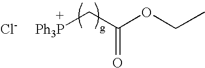

wherein g is 1 or 2, in the presence of an aprotic solvent, such as tetrahydrofuran. The reaction is refluxed for a time period between about 4 hours to about 16 hours, preferably about 10 hours. The resulting olefin is then reduced by shaking under a positive pressure of hydrogen in the presence of a catalyst, such as 20% palladium hydroxide on carbon, in the presence of a protic solvent such as ethanol. The methyl ether is deprotected according to the procedure described for reaction 2 of Preparation C.

In reaction 1 of Scheme 1, the compound of formula VI is converted to the corresponding compound of formula XVI by reacting VI with a compound of the formula, A-(C═O)—(CH₂)-A, wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 2 of Scheme 1, the compound of formula XVI is converted to the corresponding compound of formula I by reacting XVI with a compound of the formula

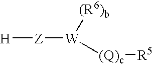

wherein Z is oxygen, which is either commercially available or is prepared according to Preparations C and D, in the presence of a base such as potassium carbonate, potassium iodide and an aprotic solvent, such as butanone. The reaction is heated to reflux for a time period between about 4 hours to about 8 hours, preferably about 6 hours.

In reaction 1 of Scheme 2, the compound of formula VI is converted to the corresponding compound of formula I by reacting VI with a compound of the formula

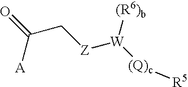

wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 1 of Scheme 3, the compound of formula VI is converted to the corresponding compound of formula XVII by reacting VI with an carboxylic acid of the formula:

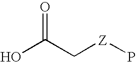

wherein Z-P is O—(C═O)—CH₃ or —NH—(C═O)—O-tBu, in the presence 4-dimethylaminopyridine, 1-(3-dimethylaminopropyl)-3-ethylcarbodiimine and a polar aprotic solvent, such as methylene chloride. In the case when Z-P is O—(C═O)—CH₃ then the resulting acetate is treated with base such as lithium hydroxide in a protic solvent such as a mixture of tetrahydrofuran, water and methanol, to give a compound of the formula XVII. In the case when Z is —NH—(C═O)—O-tBu, the resulting amide is treated with an acid, such as trifluoroacetic acid, in an aprotic solvent, such as dichloromethane to give the compound of the formula XVII.

In reaction 2 of Scheme 3, the compound of formula XVII wherein Z is oxygen, or NH, is converted to the corresponding compound of formula I where W is a (C₂-C₉)heteroaryl group, by reacting with a compound of formula Hal-W, wherein Hal is a chloro or bromo and W is an appropriately functionalized heteroaryl group, in the presence of a base, such as sodium hydride, in an aprotic solvent, such as tetrahydrofuran.

In reaction 1 of Scheme 4, the compound of formula XVI is converted to the corresponding compound of formula XVIII according to the procedure described above in reaction 2 of Scheme 1.

In reaction 2 of Scheme 4, the compound of formula XVIII is converted to the corresponding compound of formula XIX by reacting XVIII with lithium hydroxide monohydrate in the presence of methanol, tetrahydrofuran and water. The reaction mixture is stirred overnight at ambient temperature.

In reaction 3 of Scheme 4, the compound of formula XIX is converted to the corresponding amide or acylsulfonamide of formula I, by reacting XIX with an appropriate amine or sulfonamide in the presence of 4-dimethylaminopyridine, 1-(3-dimethylaminopropyl)-3-ethylcarbodiimine and a polar aprotic solvent, such as methylene chloride. The resulting reaction mixture is stirred overnight at ambient temperature.

In reaction 1 of Scheme 5, the compound of formula XVI is converted to the corresponding compound of formula XX according to the procedure described above in reaction 2 of Scheme 1.

In reaction 2 of Scheme 5, the compound of formula XX is converted to the corresponding compound of formula XXI by hydrogenating XX in the presence of a catalyst, such as platinum on carbon, and a polar protic solvent, such as ethanol. The reaction is carried out under a positive pressure of hydrogen gas between about 30 psi to about 40 psi, preferably about 35 psi, for a time period between about 15 minutes to about 1 hour, preferably 30 minutes.

In reaction 3 of Scheme 5, the compound of formula XXI is converted to the corresponding urea of formula I, by first reacting XXI with 4-nitrophenyl chloroformate in the presence of a base, such as pyridine, and a polar aprotic solvent, such as methlyene chloride, followed by reacting the intermediate so formed with an appropriate amine. The reaction mixture, so formed, is allowed to stir overnight at ambient temperature. The compound of formula XXI is reacted with an appropriate sulfonyl chloride to form the sulfonamides of formula I, in the presence of a base, such as triethylamine, and a polar aprotic solvent, such as methylene chloride. The reaction is stirred overnight at ambient temperature. To prepare cyanoguanidines of the formula I, the compound of formula XXI is first treated with sodium hydride in an aprotic solvent, such as tetrahydrofuran, followed by reacting the intermediate so formed with dimethyl-N-cyanodithio iminocarbonate. The resulting reaction mixture is heated to reflux overnight. The N-cyano-S-methyl-isothiourea intermediate is then reacted with an appropriate amine in the presence of a polar protic solvent, such as methanol, to form the cyanoguanidine of formula I. For the preparation of amides or the formula I, the compound of formula XXI is reacted with an appropriate acid in the presence of N-methylmorpholine, O-benzotriazole-1-yl-N,N, N,N′-tetramethyluronium hexafluorophosphate and a polar aprotic solvent, such as methylene chloride, to form the amide of formula I. For secondary amine formation the compound of formula XXI is reacted with an appropriate aldehyde in the presence of a reducing agent, such as sodium triacetoxyborohydride, in the presence of a polar solvent, such as methanol.

In reaction 1 of Scheme 6, the compound of formula XVI is converted to the corresponding compound of formula XXII, wherein m is 0, 1, 2, 3 or 4, according to the procedure described above in reaction 2 of Scheme 1.

In reaction 2 of Scheme 6, the compound of formula XXII is converted to the corresponding compound of formula I by reacting XXII with an appropriate amine in the presence of a 10:1 ratio solution of dichloroethane/acetic acid. The reaction mixture is stirred, at ambient temperature, for a time period between about 30 minutes to about 2 hours, preferably about 1 hour. A reducing agent, such as sodium cyanoborohydride is than added to the mixture and the reaction is allowed to stir overnight at ambient temperature. If the amine thus formed is secondary, the compound of formula I may further be reacted according to the procedure described above in reaction 3 of Scheme 5, to provide ureas, sulfonamides, cyanoguanidines, or amides.

In reaction 1 of Scheme 7, the acid compound of formula XIX is converted to the corresponding compound of formula XXIII by treating XIX with thionyl chloride neat or in an aprotic solvent, at ambient temperature, for a time period between about 1 hour to about 24 hours, preferably 1 hour. The acid chloride so formed is dissolved in a polar aprotic solvent with a compound of the formula, (H₃CO)(H₃C)NH.HCl, in the presence of an amine base, such as triethylamine. The reaction mixture is stirred, at ambient temperature, for a time period between about 1 hour to about 48 hours, preferably about 12 hours.

In reaction 2 of Scheme 7, the amide compound of formula XXIII is converted to the corresponding compound of formula I by reacting XXIII with a (C₂-C₉)heteroaryl lithium reagent in the presence of a polar aprotic solvent at a temperature between about −100° C. to ambient temperature, preferably about −78° C. The resulting reaction mixture is stirred for a time period between about 1 hour to about 24 hours, preferably about 12 hours, at a temperature between about −78° C. to about 50° C., preferably about 20° C.

In reaction 1 of Scheme 8, the compound of formula XVI is converted to the corresponding compound of formula XXIV, wherein j is 1, 2, or 3, according to the procedure described above in reaction 2 of Scheme 1.

In reaction 2 of Scheme 8, the compound of formula XXIV, wherein j is 1, 2, or 3, is converted to the corresponding compound of formula XXV, wherein j is 1, 2, or 3, according to the procedure described above in reaction 2 of Scheme 4.

In reaction 3 of Scheme 8 the compound of formula XXV, wherein j is 1, 2, or 3, is converted to the corresponding amide or acylsulfonamide of the formula I, wherein j is 1′, 2, or 3, by treating with an appropriate amine or sulfonamide according to the procedure described above in reaction 3 of Scheme 4. The compound of formula XXV, wherein j is 1, 2, or 3, is converted to other compounds of formula I according to the procedures described above for Scheme 7.

In reaction 1 of Scheme 9 the compound of formula XXIV, wherein j is 0, 1, 2, or 3, is converted to the corresponding compound of formula XXVI wherein j is 0, 1, 2, or 3, by reacting with a reducing agent, such as sodium borohydride, in a protic solvent, such as tert-butyl alcohol.

In reaction 2 of Scheme 9 the compound of formula XXVI, wherein j is 0, 1, 2, or 3, is converted to the corresponding compound of formula I by first treating with thionyl chloride, in the presence of an aprotic solvent, such as chloroform. The reaction is heated to reflux, for a time period between about 1 hour to about 10 hours, preferably about 3 hours. The resulting alkyl chloride is then treated with sodium sulfite in a polar protic solvent, such as ethanol and water, and heated to a temperature between 90° C. and 150° C., preferably around 110° C., for a time period between 10 and 20 hours, preferably 12 hours. To prepare sulfonamides or the formula I, the resulting sulfonate is treated with phosphorous pentachloride in an aprotic solvent, such as toluene, at a temperature between ambient and reflux, preferably at reflux for a time period between 1 hour and 8 hours, preferably 3 hours to give the corresponding sulfonyl chloride. The sulfonyl chloride is then reacted with an appropriate amine in a polar aprotic solvent, such as tetrahydrofuran, at ambient temperature for a time period between 3 hours and 24 hours, preferably 12 hours. The sulfonamide can be taken on further to acylsulfonamides of the formula I by treating with an acid chloride in the presence of base, such as triethylamine, in a aprotic solvent, such as dichloromethane, at ambient temperature.

Unless otherwise indicated, the pressure of each of the above reactions is not critical. Generally, the reactions are conducted at a pressure of about one to about three atmospheres, preferably at ambient pressure (about one atmosphere).

The compounds of the formula I that are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the basic compounds of this invention are readily prepared by treating the basic compound with a substantially equivalent amount of the chosen mineral or organic acid in an aqueous solvent medium or in a suitable organic solvent such as methanol or ethanol. Upon careful evaporation of the solvent, a solid salt may be obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e. 1,1′-methylene-bis-(2-hydroxy-3-naphthoate)] salts.

Those compounds of the formula I that are also acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The present invention also relates to compounds of formula I wherein any of the hydrogens may optionally be replaced by deuterium.

Unless otherwise indicated, the alkyl groups referred to herein may be linear or branched, and they may also be cyclic (e., cyclopropyl, cyclobutyl, cyclopentyl, cyclohexyl, cycloheptyl) or bicyclic (e.g., norbornanyl, bicyclo[3.2.1]octane) or contain cyclic groups. They may also contain zero to two levels of unsaturation and may be optionally substituted with 1 to 3 substituents independently selected from the group consisting of but not limited to: halo-, HO—, NC—, H₂N—, HO—(C═O)—.

Unless otherwise indicated, halogen includes fluorine, chlorine, bromine, and iodine.

(C₂-C₉)Heterocyclyl when used herein refers to, but is not limited to, pyrrolidinyl, tetrahydrofuranyl, dihydrofuranyl, tetrahydropyranyl, pyranyl, thiopyranyl, aziridinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl and chromanyl. Said (C₂-C₉)heterocyclyl ring is attached through a carbon or a nitrogen atom.

(C₂-C₉)Heteroaryl when used herein refers to, but is not limited to, furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl and may be optionally substituted with 1 to 3 substituents independently selected from the group consisting of, but not limited to: H—, HO—, halo-, (C1-C8)alkyl- optionally substituted with 1-3 fluorine atoms, (C1-C8)alkyl-O— wherein the alkyl group is optionally substituted with 1-3 fluorine atoms, HO—(C1-C8)alkyl-, NC—, H₂N—, H₂N—(C1-C8)alkyl-, HO—(C═O)—, (C1-C8)alkyl-(C═O)—, (C1-C8)alkyl-(C═O)—(C1-C8)alkyl-, H₂N—(C═O)—, H₂N—(C═O)—(C1--C8)alkyl-, H₂NSO₂—, (C1-C8)alkyl-SO₂—NH—.

Aryl when used herein refers to phenyl or naphthyl which may be optionally substituted with 1 to 3 substituents independently selected from the group consisting of but not limited to: H—, HO—, halo-, (C1-C8)alkyl- optionally substituted with 1-3 fluorine atoms, (C1-C8)alkyl-O— wherein the alkyl group is optionally substituted with 1-3 fluorine atoms, HO—(C1-C8)alkyl-, NC—, H₂N—, H₂N—(C1-C8)alkyl-, HO—(C═O)—, (C1-C8)alkyl-(C═O)—, (C1-C8)alkyl-(C═O)—(C1-C8)alkyl-, H₂N—(C═O)—, H₂N—(C═O)-(C1-C8)alkyl-, H₂NSO₂—, (C1-C8)alkyl-SO₂—NH—;

This invention also encompasses pharmaceutical compositions containing and methods of treating or preventing comprising administering prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues that are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters that are covalently bonded to the above substituents of formula I through the carbonyl carbon prodrug sidechain. This invention also provides for introduction of hydrogen isotopes (i.e., deuterium, tritium) by replacing ¹H₂ with ²H₂ or ³H₂ in the above procedure.

The compounds of this invention include all conformational isomers (e.g., cis and trans isomers. The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. In this regard, the invention includes both the E and Z configurations. The compounds of formula I may also exist as tautomers. This invention relates to the use of all such tautomers and mixtures thereof.

Compounds of the formula I and their pharmaceutically acceptable salts (hereinafter also referred to, collectively, as “the active compounds”) are potent inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic inflammatory conditions including ocular inflammation, stenosis, lung inflammation (such as chronic bronchitis, chronic, obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (co-receptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention can be assessed according to procedures know to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, are placed into the lower chambers of the Boyden chamber. A polycarbonate filter is then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber. After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of >90% to be considered a valid assay.

All of the compounds of the invention illustrated in the following examples had IC₅₀ of less than 10 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use. The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents can be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-Ig), BMS-224818 (CTLA4-Ig), RG-1046 (CTLA4-Ig), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

### EXAMPLES

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification.

### Example 1

### (Trans)-5-Chloro-2-{2-[3-(4-fluoro-phenoxy)-8-aza-bicyclo[3.2.1]oct-8-yl]-2-oxo-ethoxy}-benzamide

8-(4-Fluoro-benzyl)-8-aza-bicyclo[3.2.1]octan-3-one

A solution of 2,5-dimethoxy tetrahydrofuran (30.0 grams, 230 mmol) in 0.025 M hydrochloric acid (100 ml) was stirred overnight at 0° C. To this solution was added 4-fluoro-benzylamine hydrogen chloride (33.7 grams, 270 mmol), 3-oxo-pentanedioic acid (33.6 grams, 230 mmol), sodium acetate (10.4 grams, 120 mmol) and water (200 ml). The reaction was allowed to warm to an ambient temperature and stirred for 90 minutes, then heated to 50° C. and stirred for two hours. The reaction was then cooled to 0° C. and basified to pH=10 with a 6 N aqueous sodium hydroxide solution and extracted with ethyl acetate (3 times). The organic layers were combined, dried over magnesium sulfate, filtered and concentrated in vacuo. Silica gel chromatography gave the title compound (28.03 grams, 52% yield).

8-(4-Fluoro-benzyl)-8-aza-bicyclo[3.2.1]octan-3-ol

To a suspension of lithium aluminum hydride (1.89 grams, 49.8 mmol) in tetrahydrofuran (50 ml) at 0° C. was added a solution of 8-(4-fluoro-benzyl)-8-aza-bicyclo[3.2.1]octan-3-one (5.0 grams, 21.4 mmol) in tetrahydrofuran (50 ml). The reaction was allowed to warm to ambient temperature and stirred for three hours. The reaction was then cooled to 0° C. and quenched slowly with water. This was followed by addition of a 50% aqueous sodium hydroxide solution (50 ml) and diethyl ether (50 ml) and vigorous stirring for two hours. The reaction mixture was then filtered through celite and the filtrate was concentrated in vacuo to give the title compound (5.62 grams, >100%).

(Cis)-3-hydroxy-8-aza-bicyclo[3.2.1]octane-8-carboxylic Acid Tert-Butyl Ester and (Trans)-3-hydroxy-8-aza-bicyclo[3.2.1]octane-8-carboxylic Acid Tert-Butyl Ester

To a solution of 8-(4-fluoro-benzyl)-8-aza-bicyclo[3.2.1]octan-3-ol (5.02 grams, 21.4 mmol) in ethanol (150 ml) in a Par bottle was added carbonic acid di-tert-butyl ester (5.5 grams, 25.2 mmol) and palladium hydroxide on carbon (0.3 grams, 20% on carbon). The reaction mixture was subject to 50 psi hydrogen gas for 3 days. The reaction mixture was then filtered through a 0.54 μM filter. Concentration of the filtrate in vacuo followed by chromatography on silica gel gave the title compounds, (cis) (1.8 grams, 37% yield) and (trans) (2.3 grams, 47% yield).

(Trans)-3-(4-fluoro-phenoxy)-8-aza-bicyclo[3.2.1]octane-8-carboxylic Acid Tert-Butyl Ester

To a solution of (cis)-3-hydroxy-8-aza-bicyclo[3.2.1]octane-8-carboxylic acid tert-butyl ester (1.8 grams, 7.92 mmol) in tetrahydrofuran (40 ml) was added 4-fluoro phenol (1.35 grams, 12 mmol), triphenyl phosphine (3.15 grams, 12 mmol) followed by diethyl azidocarboxylate (1.9 ml, 12 mmol). The reaction was stirred overnight at ambient temperature and then concentrated in vacuo followed by chromatography on silica gel to give the title compound (1.55 grams, 61% yield).

(Trans)-3-(4-fluoro-phenoxy)-8-aza-bicyclo[3.2.1]octane

To a solution of (trans)-3-(4-fluoro-phenoxy)-8-aza-bicyclo[3.2.1]octane-8-carboxylic acid tert-butyl ester (0.777 g, 2.41 mmol) in dichloromethane (10 ml) was added trifluoroacetic acid (1 ml). The reaction was stirred at ambient temperature for three hours. The reaction was quenched with saturated aqueous sodium bicarbonate and extracted with dichloromethane (2 times). The organics were combined and dried over magnesium sulfate. Filtration and concentration in vacuo gave the title compound (535 mg, 100% yield).

(Trans)-5-chloro-2-{2-[3-(4-fluoro-phenoxy)-8-aza-bicyclo[3.2.1]oct-8-yl]-2-oxo-ethoxy}-benzamide

To a solution of (trans)-3-(4-fluoro-phenoxy)-8-aza-bicyclo[3.2.1]octane (118.5 mg, 0.535 mmol) in dichloromethane (5 ml) was added triethylamine, (0.115 ml, 0.825 mmol) and chloroacetyl chloride (0.050 ml, 0.655 mmol). The reaction was stirred at ambient temperature for three hours, then concentrated in vacuo. The resulting residue was then diluted in dimethyl formamide (1 ml) followed by the addition of 5-chloro-2-hydroxy-benzamide (100 mg, 0.583 mmol), potassium bicarbonate (185 mg, 1.34 mmol) and potassium iodide (100 mg, 0.602 mmol). The reaction was heated at 70° C. overnight. The reaction was then cooled, diluted with ethyl acetate and washed with water (2 times) and brine. The organics were dried over magnesium sulfate, filtered and concentrated in vacuo to give a brown oil. Silica gel chromatography gave the title compound (71.8 mg, 31% yield, LRMS M+H=433.2).

| | | LRMS |
|---|---|---|
| Example | IUPAC name | M + H |
| 2 | (5-Chloro-2-{2-[(trans)-3-(4-fluoro-phenoxy)-8-aza- | 448.2 |
| | bicyclo[3.2.1]oct-8-yl]-2-oxo-ethoxy}-phenyl)-urea | |
| 2 | (5-Chloro-2-{2-[(trans)-3-(4-fluoro-phenoxy)- | 448.1 |
| | 8-aza-bicyclo[3.2.1]oct- | |
| | 8-yl]-2-oxo-ethoxy}-phenyl)- | |
| | acetic acid | |
| 4 | N-[(5-Chloro-2-{2-[(trans)-3-(4-fluoro- | 525.1 |
| | phenoxy)-8-aza-bicyclo[3.2.1] | |
| | oct-8-yl]-2-oxo-ethoxy}-phenyl)- | |
| | acetyl]-methanesulfonamide | |
| 5 | 2-(5-Chloro-2-{2-[(trans)-3- | 447.1 |
| | (4-fluoro-phenoxy)-8-aza- | |
| | bicyclo[3.2.1]oct-8-yl]-2-oxo-ethoxy}-phenyl)- | |
| | acetamide | |

### Example 6

### (Cis)-5-chloro-2-{2-[3-(4-fluoro-phenoxy)-8-aza-bicyclo[3.2.1]oct-8-yl]-2-oxo-ethoxyy}-benzamide

(Cis)-3-(4-fluoro-phenoxy)-8-aza-bicyclo[3.2.1]octane-8-carboxylic Acid Tert-Butyl Ester

To a solution of (trans)-3-hydroxy-8-aza-bicyclo[3.2.1]octane-8-carboxylic acid tert-butyl ester (2.3 grams, 10.1 mmol) in tetrahydrofuran (50 ml) was added 4-fluoro phenol (1.75 grams, 15.6 mmol), triphenyl phosphine (4.02 grams, 15.3 mmol) and diethyl azidocarboxylate (2.4 ml, 15.2 mmol). The reaction was stirred at ambient temperature overnight. The reaction was concentrated in vacuo and chromatagraphed on silica gel to give the title compound (2.38 grams, 73% yield).

(Cis)-3-(4-fluoro-phenoxy)-8-aza-bicyclo[3.2.1]octane

To a solution of (cis)-3-(4-fluoro-phenoxy)-8-aza-bicyclo[3.2.1]octane-8-carboxylic acid tert-butyl ester (1.2 grams, 3.73 mmol) in dichloromethane (20 ml) was added trifluoroacetic acid (2 ml) The reaction was stirred at ambient temperature for three hours. The reaction was then quenched with a saturated aqueous sodium bicarbonate solution and extracted with dichloromethane (2 times). The combined organics were dried over magnesium sulfate, filtered and concentrated to give the title compound (1.07 grams, >100%).

(Cis)-2-chloro-1-[3-(4-fluoro-phenoxy)-8-aza-bicyclo[3.2.1]oct-8-yl]-ethanone

To a solution of (cis)-3-(4-fluoro-phenoxy)-8-aza-bicyclo[3.2.1]octane (1.07, 3.73 mmol) in dichloromethane (20 ml) was added triethyl amine (0.80 ml, 5.73 mmol) and chloroacetyl chloride (0.35 ml, 4.6 mmol). The reaction was stirred at ambient temperature for two hours, concentrated and chromatagraphed on silica gel to give the title compound (924 mg 83% yield).

(Cis)-5-chloro-2-{2-[3-(4-fluoro-phenoxy)-8-aza-bicyclo[3.2.1]oct-8-yl]-2-oxo-ethoxy}-benzamide

To a solution of (cis)-2-chloro-1-[3-(4-fluoro-phenoxy)-8-aza-bicyclo[3.2.1]oct-8-yl]-ethanone (402 mg, 1.35 mmol) in dimethyl formamide (4 ml) was added 5-chloro-2-hydroxy-benzamide (251 mg, 1.46 mmol), potassium carbonate (450 mg, 3.25 mmol) and potassium iodide (225 mg, 1.35 mmol). The reaction was heated at 70° C. overnight. The reaction was cooled and diluted with ethyl acetate and water. The resulting white precipitate was collected by filtration, washed with ethyl acetate, water and diethyl ether to give the title compound (376 mg, 64% yield, LRMS M+H=433.1).

The title compounds for Examples 7-41 were prepared by a method analogous to that described in Example 5.

| Ex- | | LRMS |
|---|---|---|
| ample | IUPAC name | M + H |
| 7 | 5-Chloro-2-{2-[(cis)-3-(4-fluoro-phenoxy)-8-aza- | 469.2 |
| | bicyclo[3.2.1]oct-8-yl]-2-oxo-ethoxy}- | |
| | benzenesulfonamide | |
| 8 | 2-{2-[(cis)-3-(4-Fluoro-phenoxy)-8-aza- | 429.2 |
| | bicyclo[3.2.1]oct-8-yl]-2-oxo-ethoxy}-4-methoxy- | |
| | benzamide | |
| 9 | N-Carbamoylmethyl-5-chloro-2-{2-[(cis)-3-(4-fluoro- | 490.2 |
| | phenoxy)-8-aza-bicyclo[3.2.1]oct-8-yl]-2-oxo-ethoxy}- | |
| | benzamide | |
| 10 | (5-Chloro-2-{2-[(cis)-3-(4-fluoro-phenoxy)-8-aza- | 489.3 |
| | bicyclo[3.2.1]oct-8-yl]-2-oxo-ethoxy}-benzoylamino)- | |
| | acetic acid | |
| 11 | N-(5-Chloro-2-{2-[(cis)-3-(4-fluoro-phenoxy)-8-aza- | 505.3 |
| | bicyclo[3.2.1]oct-8-yl]-2-oxo-ethoxy}-phenyl)-3- | |
| | hydroxy-3-methyl-butyramide | |
| 12 | (5-Chloro-2-{2-[(cis)-3-(4-fluoro-phenoxy)-8-aza- | 448.2 |
| | bicyclo[3.2.1]oct-8-yl]-2-oxo-ethoxy}-phenyl)-urea | |
| 13 | 5-Chloro-2-{2-[(cis)-3-(4-fluoro-phenoxy)-8-aza- | 519.2 |
| | bicyclo[3.2.1]oct-8-yl]-2-oxo-ethoxy}-N- | |
| | (2-ureido-ethyl)-benzamide | |
| 14 | 5-Chloro-2-{2-[(cis)-3-(4-fluoro-phenoxy)-8-aza- | 501.1 |
| | bicyclo[3.2.1]oct-8-yl]-2-oxo-ethoxy}-N-(2H- | |
| | tetrazol-5-yl)-benzamide | |
| 15 | 2-[4-Chloro-2-((2R)-2-methoxymethyl-pyrrolidine-1- | 531.2 |
| | carbonyl)-phenoxy]-1-[(cis)-3-(4-fluoro-phenoxy)-8- | |
| | aza-bicyclo[3.2.1]oct-8-yl]-ethanone | |
| 16 | N-(2-Amino-ethyl)-5-chloro-2-{2-[(cis)-3-(4-fluoro- | 476.2 |
| | phenoxy)-8-aza-bicyclo[3.2.1]oct-8-yl]-2-oxo-ethoxy}- | |
| | benzamide | |
| 17 | 2-[4-Chloro-2-(morpholine-4-carbonyl)-phenoxy]-1- | 503.2 |
| | (cis)-[3-(4-fluoro-phenoxy)-8-aza-bicyclo[3.2.1]oct-8- | |
| | yl]-ethanone | |
| 18 | -[4-Chloro-2-((2S)-2-methoxymethyl-pyrrolidine-1- | 531.2 |
| | carbonyl)-phenoxy]-1-[(cis)-3-(4-fluoro-phenoxy)-8- | |
| | aza-bicyclo[3.2.1]oct-8-yl]-ethanone | |
| 19 | 5-Chloro-N-(2-dimethylamino-ethyl)-2-{2-[(cis)-3-(4- | 504.3 |
| | fluoro-phenoxy)-8-aza-bicyclo[3.2.1]oct-8-yl]-2-oxo- | |
| | ethoxy}-benzamide | |
| 20 | 1-(5-Chloro-2-{2-[(cis)-3-(4-fluoro-phenoxy)-8-aza- | 547.1 |
| | bicyclo[3.2.1]oct-8-yl]-2-oxo-ethoxy}-benzoyl)- | |
| | (4R)-4-hydroxy-pyrrolidine-(2S)-2-carboxylic acid | |
| 21 | 2-[4-Chloro-2-((3R)-3-hydroxy- | 503.2 |
| | pyrrolidine-1-carbonyl)- | |
| | phenoxy]-1-[(cis)-3-(4-fluoro-phenoxy)-8-aza- | |
| | bicyclo[3.2.1]oct-8-yl]-ethanone | |
| 22 | 2-[4-Chloro-2-((3S)-3-hydroxy-pyrrolidine- | 503.2 |
| | 1-carbonyl)- | |
| | phenoxy]-1-[(cis)-3-(4-fluoro-phenoxy)-8-aza- | |
| | bicyclo[3.2.1]oct-8-yl]-ethanone | |
| 23 | 5-Chloro-2-{2-[(cis)-3-(4-fluoro-phenoxy)-8-aza- | 510.2 |
| | bicyclo[3.2.1]oct-8-yl]-2-oxo-ethoxy}-N-pyridin-2-yl- | |
| | benzamide | |
| 24 | N-(2-{2-[3-(4-Fluoro-phenoxy)-8- | 517.1 |
| | aza-bicyclo[3.2.1]oct- | |
| | 8-yl]-2-oxo-ethoxy}-5-trifluoromethyl-phenyl)- | |
| | methanesulfonamide | |
| 25 | 1-(5-Chloro-2-{2-[(cis)-3-(4-fluoro-phenoxy)-8-aza- | 547.1 |
| | bicyclo[3.2.1]oct-8-yl]-2-oxo-ethoxy}- | |
| | benzoyl)-(4R)-4- | |
| | hydroxy-pyrrolidine-(2R)-2-carboxylic acid | |
| 26 | 1-(5-Chloro-2-{2-[(cis)-3-(4-fluoro-phenoxy)-8-aza- | 547.1 |
| | bicyclo[3.2.1]oct-8-yl]-2-oxo-ethoxy}- | |
| | benzoyl)-(4S)-4- | |
| | hydroxy-pyrrolidine-(2S)-2-carboxylic acid | |
| 27 | 1-(5-Chloro-2-{2-[(cis)-3-(4-fluoro-phenoxy)-8-aza- | 546.1 |
| | bicyclo[3.2.1]oct-8-yl]-2-oxo-ethoxy}- | |
| | benzoyl)-(4S)-4- | |
| | hydroxy-pyrrolidine-(2S)-2-carboxylic acid amide | |
| 28 | 1-(5-Chloro-2-{2-[(cis)-3-(4-fluoro-phenoxy)-8-aza- | 546.1 |
| | bicyclo[3.2.1]oct-8-yl]-2-oxo-ethoxy}- | |
| | benzoyl)-(4R)-4- | |
| | hydroxy-pyrrolidine-(2S)-2-carboxylic acid amide | |
| 29 | 1-(5-Chloro-2-{2-[(cis)-3-(4-fluoro-phenoxy)-8-aza- | 546.1 |
| | bicyclo[3.2.1]oct-8-yl]-2-oxo-ethoxy}- | |
| | benzoyl)-(4R)-4- | |
| | hydroxy-pyrrolidine-(2R)-2-carboxylic acid amide | |
| 30 | N-(5-Chloro-2-{2-[(cis)-3-(4-fluoro-phenoxy)-8-aza- | 511.1 |
| | bicyclo[3.2.1]oct-8-yl]-2-oxo-ethoxy}-benzoyl)- | |
| | methanesulfonamide | |
| 31 | 2-[4-Chloro-2-(1-hydroxy-1-methyl-ethyl)-phenoxy]-1- | 430.1 |
| | (cis)-[3-(4-fluoro-phenoxy)-8-aza-bicyclo[3.2.1]oct-8- | |
| | yl]-ethanone | |
| 32 | 2-(5-Chloro-quinolin-8-yloxy)-1-[(cis)-3-(4-fluoro- | 441.2 |
| | phenoxy)-8-aza-bicyclo[3.2.1]oct-8-yl]-ethanone | |
| 33 | 2-(5-Chloro-2-{2-[(cis)-3-(4-fluoro-phenoxy)-8-aza- | 447.2 |
| | bicyclo[3.2.1]oct-8-yl]-2-oxo-ethoxy}-phenyl)- | |
| | acetamide | |
| 34 | N-[(5-Chloro-2-{2-[(cis)-3-(4-fluoro-phenoxy)-8-aza- | 525.1 |
| | bicyclo[3.2.1]oct-8-yl]-2-oxo-ethoxy}-phenyl)-acetyl]- | |
| | methanesulfonamide | |
| 35 | 5-Chloro-2-{2-[(cis)-3-(4-fluoro-phenoxy)-8-aza- | 434.1 |
| | bicyclo[3.2.1]oct-8-yl]-2-oxo-ethoxy}-benzoic acid | |
| 36 | N-(5-Chloro-2-{2-[(cis)-3-(4-fluoro-phenoxy)-8-aza- | 483.1 |
| | bicyclo[3.2.1]oct-8-yl]-2-oxo-ethoxy}-phenyl)- | |
| | methanesulfonamide | |
| 37 | (5-Bromo-2-{2-[(cis)-3-(4-fluoro-phenoxy)-8-aza- | M − H |
| | bicyclo[3.2.1]oct-8-yl]-2-oxo-ethoxy}-phenyl)-acetic | 492.2 |
| | acid | |
| 38 | 2-(5-Bromo-2-{2-[(cis)-3-(4-fluoro-phenoxy)-8-aza- | 491.1 |
| | bicyclo[3.2.1]oct-8-yl]-2-oxo-ethoxy}-phenyl)- | |
| | acetamide | |
| 39 | N-[(5-Bromo-2-{2-[(cis)-3-(4-fluoro-phenoxy)-8-aza- | 570.1 |
| | bicyclo[3.2.1]oct-8-yl]-2-oxo-ethoxy}-phenyl)-acetyl]- | |
| | methanesulfonamide | |
| 40 | 3-(5-Chloro-2-{2-[(cis)-3-(4-fluoro-phenoxy)-8-aza- | M − H |
| | bicyclo[3.2.1]oct-8-yl]-2-oxo-ethoxy}-phenyl)- | 460.3 |
| | propionic acid | |
| 41 | N-[3-(5-Chloro-2-{2-[(cis)-3- | 539.3 |
| | (4-fluoro-phenoxy)-8-aza- | |
| | bicyclo[3.2.1]oct-8-yl]-2-oxo-ethoxy}-phenyl)- | |
| | propionyl]-methanesulfonamide | |

The title compounds for Examples 42-68 were also prepared by a method analogous to that described in Example 5.

| Ex- | | LRMS |
|---|---|---|
| ample | IUPAC name | M + H |
| 42 | 1-[(cis)-3-(4-Fluoro-phenoxy)-8-aza-bicyclo[3.2.1]oct- | 356.2 |
| | 8-yl]-2-phenoxy-ethanone | |
| 43 | 2-(4-Bromo-phenoxy)-1-[(cis)-3-(4-fluoro-phenoxy)-8- | 434.1 |
| | aza-bicyclo[3.2.1]oct-8-yl]-ethanone | |
| 44 | 1-[(cis)-3-(4-Fluoro-phenoxy)-8-aza-bicyclo[3.2.1]oct- | 424.2 |
| | 8-yl]-2-(4-trifluoromethyl-phenoxy)-ethanone | |
| 45 | 1-[(cis)-3-(4-Fluoro-phenoxy)-8-aza-bicyclo[3.2.1]oct- | 370.2 |
| | 8-yl]-2-p-tolyloxy-ethanone | |
| 46 | 2-(4-Chloro-phenoxy)-1-(cis)-[3-(4-fluoro-phenoxy)-8- | 390.2 |
| | aza-bicyclo[3.2.1]oct-8-yl]-ethanone | |
| 47 | 2-(2-Acetyl-4-chloro-phenoxy)-1-[(cis)-3-(4-fluoro- | 432.1 |
| | phenoxy)-8-aza-bicyclo[3.2.1]oct-8-yl]-ethanone | |
| 48 | 5-Chloro-2-{2-[(cis)-3-(4-fluoro-phenoxy)-8-aza- | 447.2 |
| | bicyclo[3.2.1]oct-8-yl]-2-oxo- | |
| | ethoxy}-N-methyl-benzamide | |
| 49 | 5-Bromo-2-{2-[(cis)-3-(4-fluoro-phenoxy)-8-aza- | 479.2 |
| | bicyclo[3.2.1]oct-8-yl]-2-oxo-ethoxy}-benzamide | |
| 50 | 2-(4-Chloro-2-hydroxymethyl-phenoxy)-1-[(cis)-3-(4- | 420.2 |
| | fluoro-phenoxy)-8-aza- | |
| | bicyclo[3.2.1]oct-8-yl]-ethanone | |
| 51 | 2-(4-Bromo-2-hydroxymethyl-phenoxy)-1-(cis)-[3-(4- | 464.1 |
| | fluoro-phenoxy)-8-aza- | |
| | bicyclo[3.2.1]oct-8-yl]-ethanone | |
| 52 | 2-(4-Chloro-2-hydroxy-phenoxy)-1-[(cis)-3-(4-fluoro- | 406.4 |
| | phenoxy)-8-aza-bicyclo[3.2.1]oct-8-yl]-ethanone | |
| 53 | (5-Chloro-2-{2-[(cis)-3-(4-fluoro-phenoxy)-8-aza- | 462.3 |
| | bicyclo[3.2.1]oct-8-yl]-2-oxo-ethoxy}-phenoxy)-acetic | |
| | acid | |
| 54 | 2-(4-Bromo-2-hydroxy-phenoxy)-1-[(cis)-3-(4-fluoro- | 450.1 |
| | phenoxy)-8-aza-bicyclo[3.2.1]oct-8-yl]-ethanone | |
| 55 | 5-Chloro-2-{2-[(cis)-3-(4-fluoro-phenoxy)-8-aza- | 477.2 |
| | bicyclo[3.2.1]oct-8-yl]-2-oxo-ethoxy}-N-(2-hydroxy- | |
| | ethyl)-benzamide | |
| 56 | 5-Chloro-2-{2-[(cis)-3-(4-fluoro-phenoxy)-8- | 491.2 |
| | azabicyclo[3.2.1]oct-8-yl]-2-oxo-ethoxy}- | |
| | N-(3-hydroxy- | |
| | propyl)-benzamide | |
| 57 | 4-(5-Chloro-2-{2-[(cis)-3-(4-fluoro-phenoxy)-8-aza- | 519.3 |
| | bicyclo[3.2.1]oct-8-yl]-2-oxo-ethoxy}-phenoxy)- | |
| | pyrrolidine-(2S)-2-carboxylic acid | |
| 58 | (2S)-2-Amino-4-(5-chloro-2-{2-[(cis)-3-(4-fluoro- | 507.3 |
| | phenoxy)-8-aza-bicyclo[3.2.1]oct-8-yl]-2-oxo-ethoxy}- | |
| | phenoxy)-butyric acid | |
| 59 | (5-Chloro-2-{2-[(cis)-3-(4-fluoro-phenoxy)-8-aza- | 483.1 |
| | bicyclo[3.2.1]oct-8-yl]-2-oxo-ethoxy}-phenyl)- | |
| | methanesulfonamide | |
| 60 | N-Acetyl-C-(5-chloro-2-{2-[(cis)-3- | 525.1 |
| | (4-fluoro-phenoxy)- | |
| | 8-aza-bicyclo[3.2.1]oct-8-yl]-2-oxo-ethoxy}-phenyl)- | |
| | methanesulfonamide | |
| 61 | (5-Bromo-2-{2-[(cis)-3-(4-fluoro-phenoxy)-8-aza- | M − H |
| | bicyclo[3.2.1]oct-8-yl]-2-oxo-ethoxy}-phenyl)- | 527.2 |
| | methanesulfonamide | |
| 62 | N-Acetyl-C-(5-bromo-2-{2-[(cis)-3- | m − H |
| | (4-fluoro-phenoxy)- | 569.1 |
| | 8-aza-bicyclo[3.2.1]oct-8-yl]-2-oxo-ethoxy}-phenyl)- | |
| | methanesulfonamide | |
| 63 | C-(5-Chloro-2-{2-[(cis)-3-(4-fluoro-phenoxy)-8-aza- | 569.3 |
| | bicyclo[3.2.1]oct-8-yl]-2-oxo-ethoxy}-phenyl)-N-(2- | |
| | hydroxy-2-methyl-propionyl)-methanesulfonamide | |
| 64 | C-(5-Chloro-2-{2-[(cis)-3-(4-fluoro-phenoxy)-8-aza- | 541.3 |
| | bicyclo[3.2.1]oct-8-yl]-2-oxo-ethoxy}-phenyl)-N- | |
| | hydroxyacetyl-methanesulfonamide | |
| 65 | C-(5-Chloro-2-{2-[(cis)-3-(4-fluoro-phenoxy)-8-aza- | 541.1 |
| | bicyclo[3.2.1]oct-8-yl]-2-oxo-ethoxy}-phenyl)-N- | |
| | (methoxycarbonyl)-methanesulfonamide | |
| 66 | C-(5-Chloro-2-{2-[(cis)-3-(4-fluoro-phenoxy)-8-aza- | 567.3 |
| | bicyclo[3.2.1]oct-8-yl]-2-oxo-ethoxy}-phenyl)-N-(1- | |
| | hydroxy-cyclopropanecarbonyl)-methanesulfonamide | |
| 67 | C-(5-Chloro-2-{2-[(cis)-3-(4-fluoro-phenoxy)-8-aza- | 555.4 |
| | bicyclo[3.2.1]oct-8-yl]-2-oxo-ethoxy}-phenyl)-N- | |
| | methoxyacetyl-methanesulfonamide | |
| 68 | (5-Chloro-2-{2-[(cis)-3-(4-fluoro-phenoxy)-8-aza- | M − H |
| | bicyclo[3.2.1]oct-8-yl]-2-oxo-ethoxy}-phenyl)- | 482.3 |
| | methanesulfonic acid | |

### Example 69

### (Cis)-5-Chloro-2-{(2-[3-(4-fluoro-phenoxy)-8-aza-bicyclo[3.2.1]oct-8-yl]-2-oxo-ethylamino}-nicotinamide

(Cis)-{2-[3-(4-fluoro-phenoxy)-8-aza-bicyclo[3.2.1]oct-8-yl]-2-oxo-ethyl}-carbamic Acid Tert-Butyl Ester

To a solution of (cis)-3-(4-fluoro-phenoxy)-8-aza-bicyclo[3.2.1]octane (790 mg, 3.57 mmol) in dichloromethane (20 ml) was added tert-butoxycarbonylamino-acetic acid (688 mg, 3.93 mmol), (3-(dimethylamino)propyl)ethyl carbodiimide hydrochloride (1.03 grams, 5.36 mmol), [1,2,3]triazolo[4,5-b]pyridin-3-ol (627 mg, 4.64 mmol) and triethyl amine (1.48 ml, 10.7 mmol). The reaction was stirred at ambient temperature overnight. The reaction was then diluted with saturated aqueous sodium bicarbonate and extracted with dichloromethane (3 timesx). The organic layers were combined, dried over magnesium sulfate, filtered and concentrated in vacuo. Purification by silica gel chromatography gave the title compound (449.1 mg, 74% yield).

(Cis)-2-amino-1-[3-(4-fluoro-phenoxy)-8-aza-bicyclo[3.2.1]oct-8-yl]-ethanone

To a solution of (cis)-{2-[3-(4-fluoro-phenoxy)-8-aza-bicyclo[3.2.1]oct-8-yl]-2-oxo-ethyl}-carbamic acid tert-butyl ester (888 mg, 2.35 mmol) in dichloromethane (15 ml) was added trifluoroacetic acid (7 ml). The reaction was stirred at ambient temperature for three hours. The reaction was basified with 50% aqueous sodium hydroxide and extracted with dichloromethane (2 times) and ethyl acetate The organic layers were combined, dried over magnesium sulfate, filtered and concentrated in vacuo to give the title compound (619 mg, 95% yield).

(Cis)-5-chloro-2-{2-[3-(4-fluoro-phenoxy)-8-aza-bicyclo[3.2.1]oct-8-yl]-2-oxo-ethylamino}-nicotinamide

To a solution of (cis)-2-amino-1-[3-(4-fluoro-phenoxy)-8-aza-bicyclo[3.2.1]oct-8-yl]-ethanone (70 mg, 0.252 mmol) in dimethyl formamide (1 ml) was added 2,5-dichloro-nicotinamide (53 mg, 0.277 mmol), and triethyl amine (42 μl, 0.302 mmol). The reaction was stirred at 80° C. overnight. The reaction was then cooled, diluted with water and extracted with ethyl acetate (3 times). The organic layers were combined, dried over sodium sulfate and concentrated in vacuo. Purification by silica gel chromatography gave the title compound (24.1 mg, 20% yield, LRMS M+H 433.1).

The title compounds for Examples 70-88 were prepared by a method analogous to that described in Example 69.

| Ex- | | LRMS |
|---|---|---|
| ample | IUPAC name | M + H |
| 70 | (cis)-5-Chloro-N-(2-dimethylamino-ethyl)-2-{2-[3-(4- | 504.2 |
| | fluoro-phenoxy)-8-aza-bicyclo[3.2.1]oct-8-yl]-2-oxo- | |
| | ethylamino}-nicotinamide | |
| 71 | (cis)-N-(2-Amino-ethyl)-5-chloro-2-{2-[3-(4-fluoro- | 476.2 |
| | phenoxy)-8-aza-bicyclo[3.2.1]oct-8-yl]-2-oxo- | |
| | ethylamino}-nicotinamide | |
| 72 | [(cis)-(5-Chloro-2-{2-[3-(4-fluoro-phenoxy)-8-aza- | 491.1 |
| | bicyclo[3.2.1]oct-8-yl]-2-oxo-ethylamino}-pyridine-3- | |
| | carbonyl)-amino]-acetic acid | |
| 73 | 2-[5-Chloro-3-(morpholine-4-carbonyl)-pyridin-2- | 503.2 |
| | ylamino]-1-[(cis)-3-(4-fluoro-phenoxy)-8-aza- | |
| | bicyclo[3.2.1]oct-8-yl]-ethanone | |
| 74 | 2-[5-Chloro-3-((3S)-3-hydroxy-pyrrolidine- | M − H |
| | 1carbonyl)-pyridin-2-ylamino]-1-[(cis)-3-(4-fluoro- | 501.3 |
| | phenoxy)-8-aza-bicyclo[3.2.1]oct-8-yl]-ethanone | |
| 75 | 2-[5-Chloro-3-((3R)-3-hydroxy-pyrrolidine-1- | 503.2 |
| | carbonyl)-pyridin-2-ylamino]-1-[(cis)-3-(4-fluoro- | |
| | phenoxy)-8-aza-bicyclo[3.2.1]oct-8-yl]-ethanone | |
| 76 | 2-[5-Chloro-3-((2S)-2-methoxymethyl-pyrrolidine-1- | 531.2 |
| | carbonyl)-pyridin-2-ylamino]-1-[(cis)-3-(4-fluoro- | |
| | phenoxy)-8-aza-bicyclo[3.2.1]oct-8-yl]-ethanone | |
| 77 | 2-[5-Chloro-3-((2R)-2-methoxymethyl-pyrrolidine-1- | 531.2 |
| | carbonyl)-pyridin-2-ylamino]-1-[(cis)-3-(4-fluoro- | |
| | phenoxy)-8-aza-bicyclo[3.2.1]oct-8-yl]-ethanone | |
| 78 | 1-(5-Chloro-2-{2-[(cis)-3-(4-fluoro-phenoxy)-8-aza- | 546.1 |
| | bicyclo[3.2.1]oct-8-yl]-2-oxo-ethylamino}-pyridine-3- | |
| | carbonyl)-(4R)-4-hydroxy-pyrrolidine-(2S)-2- | |
| | carboxylic acid amide | |
| 79 | 1-(5-Chloro-2-{2-[(cis)-3-(4-fluoro-phenoxy)-8-aza- | 546.1 |
| | bicyclo[3.2.1]oct-8-yl]-2-oxo-ethylamino}-pyridine-3- | |
| | carbonyl)-(4R)-4-hydroxy-pyrrolidine-(2R)-2- | |
| | carboxylic acid amide | |
| 80 | 1-(5-Chloro-2-{2-[(cis)-3-(4-fluoro-phenoxy)-8-aza- | 546.1 |
| | bicyclo[3.2.1]oct-8-yl]-2-oxo-ethylamino}-pyridine-3- | |
| | carbonyl)-(4S)-4-hydroxy-pyrrolidine-(2S)-2- | |
| | carboxylic acid amide | |
| 81 | 1-(5-Chloro-2-{2-[(cis)-3-(4-fluoro-phenoxy)-8-aza- | 547.1 |
| | bicyclo[3.2.1]oct-8-yl]-2-oxo-ethylamino}-pyridine-3- | |
| | carbonyl)-(4R)-4-hydroxy-pyrrolidine-(2S)-2- | |
| | carboxylic acid | |
| 82 | 1-(5-Chloro-2-{2-[(cis)-3-(4-fluoro-phenoxy)-8-aza- | 547.1 |
| | bicyclo[3.2.1]oct-8-yl]-2-oxo-ethylamino}-pyridine-3- | |
| | carbonyl)-(4S)-4-hydroxy-pyrrolidine-(2S)-2- | |
| | carboxylic acid | |
| 83 | 1-(5-Chloro-2-{2-[(cis)-3-(4-fluoro-phenoxy)-8-aza- | 547.1 |
| | bicyclo[3.2.1]oct-8-yl]-2-oxo-ethylamino}-pyridine-3- | |
| | carbonyl)-(4R)-4-hydroxy-pyrrolidine-(2R)-2- | |
| | carboxylic acid | |
| 84 | (cis)-N-Carbamoylmethyl-5-chloro-2-{2-[3-(4-fluoro- | 490.2 |
| | phenoxy)-8-aza-bicyclo[3.2.1]oct-8-yl]-2-oxo- | |
| | ethylamino}-nicotinamide | |
| 85 | (cis)-5-Chloro-2-{2-[3-(4-fluoro-phenoxy)-8-aza- | M − H |
| | bicyclo[3.2.1]oct-8-yl]-2-oxo-ethylamino}-nicotinic | 432.2 |
| | acid | |
| 86 | 5-Chloro-2-{2-[(cis)-3-(4-fluoro-phenoxy)-8-aza- | 511.2 |
| | bicyclo[3.2.1]oct-8-yl]-2-oxo-ethylamino}-N- | |
| | pyrimidin-4-yl-nicotinamide | |
| 87 | N-(5-Chloro-2-{2-[(cis)-3-(4-fluoro-phenoxy)-8-aza- | 511.2 |
| | bicyclo[3.2.1]oct-8-yl]-2-oxo-ethylamino}-pyridine-3- | 513.2 |
| | carbonyl)-methanesulfonamide | |
| 88 | 5-Chloro-2-{2-[(cis)-3-(4-fluoro-phenoxy)-8-aza- | 510.1 |
| | bicyclo[3.2.1]oct-8-yl]-2oxo-ethylamino}-N-pyridin-2- | 512.1 |
| | yl-nicotinamide | |

### Example 89

### 5-Chloro-2-{2-[(cis)-3-(4-fluoro-phenoxy)-8-aza-bicyclo[3.2.1]oct-8-yl]-2-oxo-ethoxy}-nicotinamide

Acetic Acid 2-[(cis)-3-(4-fluoro-phenoxy)-8-aza-bicyclo[3.2.1]oct-8-yl]-2-oxo-ethyl Ester

To a solution of (cis)-3-(4-fluoro-phenoxy)-8-aza-bicyclo[3.2.1]octane (920 mg, 3.3 mmol) in dichloromethane (15 ml) at 0° C. was added triethylamine (0.69 ml, 4.95 mmol) and acetic acid chlorocarbonylmethyl ester (0.425 ml, 3.95 mmol). The reaction was allowed to warm to ambient temperature and stirred for two hours. The reaction was then diluted with dichloromethane and washed with a 0.2 M aqueous hydrochloric acid solution. The organic layer was separated, dried over magnesium sulfate, filtered and concentrated in vacuo to give the title compound (1.08 g, 100% yield).

(Cis)-1-[3-(4-fluoro-Phenoxy)-8-aza-bicyclo[3.2.1]oct-8-yl]-2-hydroxy-ethanone

To a solution of acetic acid 2-[(cis)-3-(4-fluoro-phenoxy)-8-aza-bicyclo[3.2.1]oct-8-yl]-2-oxo-ethyl ester in tetrahydrofuran (6 ml), methanol (6 ml) and water (3 ml) was added lithium hydroxide monohydrate (203 mg, 4.84 mmol). The reaction was stirred at ambient temperature for 30 minutes. The reaction was then diluted with water and extracted with ethyl acetate (2 timesx). The organic layers were combined and washed with saturated aqueous sodium chloride, dried over magnesium sulfate, filtered and concentrated in vacuo to give the title compound (803 mg, 87% yield).

5-chloro-2-{2-[(cis)-3-(4-fluoro-phenoxy)-8-aza-bicyclo[3.2.1]oct-8-yl]-2-oxo-ethoxy}-nicotinamide

To a solution of 1-[(cis)-3-(4-fluoro-phenoxy)-8-aza-bicyclo[3.2.1]oct-8-yl]-2-hydroxy-ethanone (101 mg, 0.358 mmol) in toluene (2 ml) at 0° C. was added sodium hydride (20 mg, 0.5 mmol, 60% dispersion in mineral oil). The reaction was stirred for 15 minutes at 0° C. followed by addition of 2,5-dichloro-nicotinamide. The reaction was allowed to warm to ambient temperature and stirred overnight. The reaction was then diluted with water and ethyl acetate precipitating a white solid. The solid was collected by filtration and washed with water, ethanol and diethyl ether, then air dried to give the title compound (63.8 g, 41% yield, LRMS M+H=434.2).

The title compounds for Example 90-94 were prepared by a method analogous to that described in 89.

| | | LRMS |
|---|---|---|
| Example | IUPAC name | M + H |
| 90 | N-Acetyl-5-chloro-2-{2-[(cis)-3-(4-fluoro- | 476.0 |
| | phenoxy)-8-aza-bicyclo[3.2.1]oct-8-yl]-2-oxo- | |
| | ethoxy}-nicotinamide | |
| 91 | 2-(3-Amino-5-chloro-pyridin-2-yloxy)-1-[(cis)-3-(4- | 406.2 |
| | fluoro-phenoxy)-8-aza-bicyclo[3.2.1]oct-8-yl]- | |
| | ethanone | |
| 92 | (5-Chloro-2-{2-[(cis)-3-(4-fluoro-phenoxy)-8-aza- | 449.2 |
| | bicyclo[3.2.1]oct-8-yl]-2-oxo-ethoxy}-pyridin-3-yl)- | |
| | urea | |
| 93 | 2-Amino-N-(5-chloro-2-{2-[(cis)-3-(4-fluoro- | 463.2 |
| | phenoxy)-8-aza-bicyclo[3.2.1]oct-8-yl]-2-oxo- | |
| | ethoxy}-pyridin-3-yl)-acetamide | |
| 94 | N-Acetyl-5-chloro-2-{2-[(cis)-3-(4-fluoro- | 506.2 |
| | phenoxy)-8-aza-bicyclo[3.2.1]oct-8-yl]-2-oxo- | |
| | ethoxy}-nicotinamide | |

### Example 95

### (Cis)-5-Chloro-2-{2-[3-(4-fluoro-phenylamino)-8-aza-bicyclo[3.2.1]oct-8-yl]-2-oxo-ethoxy}-benzamide

8-Benzyl-8-aza-bicyclo[3.2.1]octan-3-one

To a solution of 0.025 M aqueous hydrochloric acid (100 ml) at 0° C. was added 2,5-dimethoxy-tetrahydrofuran (30 ml 231 mmol). The reaction was stirred at 0° C. overnight. The reaction was then diluted with water (200 ml) and benzyl amine hydrochloride (40 grams, 278 mmol), 3-oxo-pentanedioic acid (33.7 grams, 231 mmol), and sodium acetate (10.7 grams, 130 mmol) were added. The reaction was stirred for 5 minutes at 0° C., warmed to ambient temperature and stirred for 90 minutes, then heated to 50° C. for two hours, cooled to 0° C. and basified to pH=10 with 50% aqueous sodium hydroxide (14 ml). The reaction mixture was extracted with ethyl acetate (3 times) and the organic layers were combined and washed with a saturated sodium chloride solution, dried over magnesium sulfate, filtered and concentrated in vacuo to give a brown oil. Silica gel chromatography gave the title compound (33.46 grams, 67% yield).

(Cis)-(8-Benzyl-8-aza-bicyclo[3.2.1]oct-3-yl)-(4-fluoro-phenyl)-amine

To a solution of 8-benzyl-8-aza-bicyclo[3.2.1]octan-3-one (3.09 grams, 14.35 mmol) in dichloroethane was added 4-fluoro-phenyl amine (1.4 ml, 14.78 mmol), acetic acid (1.2 ml, 20.96 mmol) and sodium triacetoxyborohydride (4.64 grams, 21.89 mmol). The reaction was stirred at ambient temperature for four days. The reaction was then quenched with 1 M aqueous sodium hydroxide and stirred for 10 minutes. The reaction mixture was extracted with dichloromethane (2 times), the combined organic layers were dried over magnesium sulfate, filtered and concentrated in vacuo to give a yellow solid. Silica gel chromatography gave the title compound (3.28 grams, 73% yield).

(Cis)-(8-Aza-bicyclo[3.2.1]oct-3-yl)-(4-fluoro-phenyl)-amine

To a solution of (cis)-(8-benzyl-8-aza-bicyclo[3.2.1]oct-3-yl)-(4-fluoro-phenyl)-amine (3.28 grams, 10.56 mmol) in methanol (80 ml) was added ammonium formate (3.3 grams, 52.3 mmol) and palladium on carbon (300 mg, 10% on carbon). The reaction was heated at reflux for two hours. The reaction was cooled, filtered through a 0.45 μM filter and concentrated in vacuo. The resulting residue was taken up in dichloromethane and washed with saturated aqueous sodium bicarbonate solution. The organic layer was separated, dried over magnesium sulfate, filtered and concentrated to give the title compound (1.54 grams, 66% yield).

(Cis)-2-Chloro-1-[3-(4-fluoro-phenylamino)-8-aza-bicyclo[3.2.1]oct-8-yl]-ethanone

To a solution of (cis)-(8-aza-bicyclo[3.2.1]oct-3-yl)-(4-fluoro-phenyl)-amine (503 mg, 2.28 mmol) in dichloromethane at 0° C. was added triethyl amine (0.350 ml, 2.51 mmol), and chloroacetyl chloride (0.175 ml, 2.29 mmol). The reaction was stirred at 0° C. for thirty minutes. Silica gel chromatography gave the title compound (404 mg, 60% yield).

(Cis)-5-Chloro-2-{2-[3-(4-fluoro-phenylamino)-8-aza-bicyclo[3.2.1]oct-8-yl]-2-oxo-ethoxy}-benzamide

To a solution of (cis)-2-chloro-1-[3-(4-fluoro-phenylamino)-8-aza-bicyclo[3.2.1]oct-8-yl]-ethanone (51.5 mg, 0.173 mmol) in dimethylformamide (0.5 ml) was added 5-chloro-2-hydroxy-benzamide (35 mg, 0.203 mmol), potassium carbonate (61 mg, 0.44 mmol) and potassium iodide (31 mg, 0.186 mmol). The reaction was heated at 80° C. overnight. The reaction was cooled, diluted with water and extracted with ethyl acetate (2 times). The organic layers were combined, dried over magnesium sulfate, filtered and concentrated to give a solid. The solid was triturated in diethyl ether and the liquids were decanted off to give the title compound (65.9 mg, 88% yield, LRMS M+H 432.2).

### Example 96

### 5-Chloro-2-{2-[(trans)-7-(4-fluoro-phenoxy)-3-oxa-9-aza-bicyclo[3.3.1]non-9-yl]-2-oxo-ethoxy}-benzamide

2-(2,2-Diethoxy-ethoxy)-1,1-diethoxy-ethane

To a suspension of sodium hydride (3.0 g, 60% dispersion in mineral oil, 125 mmol) in xylenes under nitrogen was added 2,2-diethoxy-ethanol (15.3 g, 114 mmol) dropwise via cannula. The reaction was heated to reflux for two hours, cooled to ambient temperature followed by addition of 2-bromo-1,1-diethoxy-ethane (25.6 mL, 170 mmol) dropwise. The reaction was then heated at reflux overnight. The xylenes were distilled off under atmospheric pressure. The title compound was distilled off under vacuum (6 mm Hg) at 120° C. (12.0 grams, 42% yield).

9-Benzyl-9-aza-bicyclo[3.3.1]nonane-3,7-dione

A solution of 2-(2,2-diethoxy-ethoxy)-1,1-diethoxy-ethane (12.0 grams, 47.9 mmol) in acetic acid (2.8 ml) and water (12 ml) was heated at reflux for one hour, cooled to an ambient temperature and stirred overnight. To the reaction mixture was then added benzyl amine hydrochloride (6.9 grams, 47.9 mmol), 3-oxo-pentanedioic acid (5.48 g, 39.9 mmol), sodium acetate (2.7 grams, 20 mmol) and water (24 ml). The reaction was stirred for one hour, heated at 50° C. for three hours, cooled to ambient temperature and then basified with 50% aqueous sodium hydroxide. The reaction mixture was extracted with ethyl acetate (3). The organic layers were combined, dried over sodium sulfate, filtered and concentrated in vacuo. Silica gel chromatography gave the title compound (4.23 grams, 38% yield).

(Cis)-9-Benzyl-7-hydroxy-9-aza-bicyclo[3.3.1]nonan-3-one

To a solution of 9-benzyl-9-aza-bicyclo[3.3.1]nonane-3,7-dione (855 mg, 3.7 mmol) in tetrahydrofuran (11 ml) at 0° C. was added lithium borohydride (5.5 ml, 2 M solution in THF, 11.1 mmol) dropwise. The reaction was allowed to warm to ambient temperature and stirred for 21 hours. The reaction was then cooled to 0° C. and quenched with water (1 ml) followed by 2M aqueous hydrochloric acid (1 ml). The reaction mixture was concentrated in vacuo, treated with hydrochloric acid and refluxed for one hour. The reaction was cooled to ambient temperature, basified with 50% aqueous sodium hydroxide, and extracted with dichloromethane (3× times). The combined organic layers were dried over sodium sulfate, filtered and concentrated in vacuo to give the title compound (868 mg, 100% yield).

(Cis)-3-Hydroxy-7-oxo-9-aza-bicyclo[3.3.1]nonane-9-carboxylic Acid Tert-Butyl Ester

To a solution of 9-benzyl-7-hydroxy-9-aza-bicyclo[3.3.1]nonan-3-one (860 mg, 3.69 mmol) in ethanol (4 ml) was added palladium hydroxide on carbon (430 mg, 20% on carbon). The reaction mixture was then subject to 50 psi hydrogen gas for 27.5 hours. The reaction mixture was filtered through a nylon filter and concentrated in vacuo. Silica gel chromatography gave the title compound (701 mg, 78% yield).

(Trans)-3-(4-fluoro-phenoxy)-7-oxo-9-aza-bicyclo[3.3.1]nonane-9-carboxylic Acid Tert-Butyl Ester

To a solution of (cis)-3-hydroxy-7-oxo-9-aza-bicyclo[3.3.1]nonane-9-carboxylic acid tert-butyl ester (350 mg, 1.44 mmol) in tetrahydrofuran (7 ml) was added 4-flurophenol (242 mg, 2.16 mmol), triphenyl phosphine (566 mg, 2.16 mmol) and diethyl azidocarboxylate (0.340 ml, 2.16 mmol). The reaction is stirred at ambient temperature for 18 hours, concentrated in vacuo and silica gel chromatography gave the title compound (56.4 mg, 12% yield).

(Trans)-7-(4-Fluoro-phenoxy)-9-aza-bicyclo[3.3.1]nonan-3-one

To a solution of (trans)-3-(4-fluoro-phenoxy)-7-oxo-9-aza-bicyclo[3.3.1]nonane-9-carboxylic acid tert-butyl ester (48 mg, 0.142 mmol) in dichloromethane (1 ml) was added trifluoroacetic acid (0.5 ml). The reaction was stirred for 2.5 hrs at ambient temperature. The reaction was then diluted with saturated aqueous sodium bicarbonate, extracted with dichloromethane (3 times), dried over sodium sulfate, filtered and concentrated to give the title compound (32 mg, 95% yield).

(Trans)-5-Chloro-2-{(2-[3-(4-fluoro-phenoxy)-7-oxo-9-aza-bicyclo[3.3.1]non-9-yl]-2-oxo-ethoxy}-benzamide

To a solution of (trans)-7-(4-fluoro-phenoxy)-9-aza-bicyclo[3.3.1]nonan-3-one (32 mg, 0.135 mmol) in dichloromethane at 0° C. was added triethyl amine (28 μl, 0.202 mmol) and choroacetyl chloride (12 μL, 0.148 mmol). The reaction was stirred for one hour and then concentrated in vacuo. The resulting residue was dissolved in dimethyl formamide (0.5 ml). To this was added 5-chloro-2-hydroxy-benzamide (25 mg, 0.149 mmol), potassium carbonate (37 mg, 0.270 mmol) and potassium iodide (22 mg, 0.135 mmol). The reaction was heated at 80° C. overnight, cooled to ambient temperature, diluted with water and extracted with ethyl acetate (3 times). The organic-layers were combined, dried over sodium sulfate, filtered and concentrated in vacuo. Silica gel chromatography gave the title compound (12.3 mg, 20% yield, LRMS M+H=449.3).

The title compounds for Examples 97-98 were prepared by a method analogous to that described in Example 96.

| | | LRMS |
|---|---|---|
| Example | IUPAC name | M + H |
| 97 | (5-Chloro-2-{2-[(trans)-7-(4-fluoro-phenoxy)-3- | 464.1 |
| | oxa-9-aza-bicyclo[3.3.1]non-9-yl]-2-oxo- | |
| | ethoxy}-phenyl)-acetic acid | |
| 98 | N-[(5-Chloro-2-{2-[(trans)-7-(4-fluoro- | 541.0 |
| | phenoxy)-3-oxa-9-aza-bicyclo[3.3.1]non-9-yl]- | |
| | 2-oxo-ethoxy}-phenyl)-acetyl]- | |
| | methanesulfonamide | |

### Example 99

### N-[(5-Chloro-2-{2-[(cis)-3-(4-fluoro-phenoxy)-8-aza-bicyclo[3.2.1]oct-8-yl]-2-oxo-ethoxy}-benzyloxy)-acetyl]-methanesulfonamide

5-Chloro-2-{2-[(cis)-3-(4-fluoro-phenoxy)-8-aza-bicyclo[3.2.1]oct-8-yl]-2-oxo-ethoxyy}-benzaldehyde

To a solution of 2-chloro-1-[(cis)-3-(4-fluoro-phenoxy)-8-aza-bicyclo[3.2.1]oct-8-yl]-ethanone (390 mg, 1.31 mmol) in dimethyl formamide (4 ml) was added 5-chloro-2-hydroxy-benzaldehyde (256 mg, 1.44 mmol), potassium carbonate (362 mg, 2.62 mmol) and potassium iodide (217 mg, 1.31 mmol). The reaction was stirred at 80° C. overnight. The reaction was then cooled, diluted with water and extracted with ethyl acetate. The combined organic layers were dried over sodium sulfate, filtered and concentrated in vacuo. Silica gel chromatography gave the title compound (489 mg, 89% yield).

2-(4-Chloro-2-hydroxymethyl-phenoxy)-1-[(cis)-3-(4-fluoro-phenoxy)-8-aza-bicyclo[3.2.1]oct-8-yl]-ethanone

To a solution of 5-chloro-2-{2-[(cis)-3-(4-fluoro-phenoxy)-8-aza-bicyclo[3.2.1]oct-8-yl]-2-oxo-ethoxy}-benzaldehyde (480 mg, 1.15 mg) in methanol (15 ml) was added resin bound borohydride (1.2 g, 2.87 mmol). The reaction was stirred at ambient temperature for 21 hours, then filtered and concentrated in vacuo to give the title compound (445.1 mg, 92% yield).

(5-Chloro-2-{2-[(cis)-3-(4-fluoro-phenoxy)-8-aza-bicyclo[3.2.1]oct-8-yl]-2-oxo-ethoxy}-benzyloxy)-acetic Acid Tert-Butyl Ester

To a solution of sodium hydride (26 mg, 1.07 mmol) in tetrahydorfuran (3.5 ml) at 0° C. was added 2-(4-chloro-2-hydroxymethyl-phenoxy)-1-[(cis)-3-(4-fluoro-phenoxy)-8-aza-bicyclo[3.2.1]oct-8-yl]-ethanone (300 mg, 0.714 mmol) and bromo-acetic acid tert-butyl ester (26 mg, 2.14 mmol). The reaction was allowed to warm to ambient temperature and stirred for 17 hours. The reaction was quenched with water and extracted with ethyl acetate (3 times tmes). The combined organic layers were dried over sodium sulfate, filtered and concentrated in vacuo. Silica gel chromatography gave the title compound (278.3 mg, 73% yield).

(5-Chloro-2-{2-[(cis)-3-(4-fluoro-phenoxy)-8-aza-bicyclo[3.2.1]oct-8-yl]-2-oxo-ethoxy}-benzyloxy)-acetic Acid

To a solution of (5-chloro-2-{2-(cis)-[3-(4-fluoro-phenoxy)-8-aza-bicyclo[3.2.1]oct-8-yl]-2-oxo-ethoxy}-benzyloxy)-acetic acid tert-butyl ester (270 mg, 0.560 mmol) in dichloromethane (5 ml) was added trifluoroacetic acid (1 ml). The reaction was stirred at ambient temperature overnight. The reaction was diluted with 0.2 N aqueous hydrochloric acid and extracted with dichloromethane (3×). The combined organic layers were dried over sodium sulfate, filtered and concentrated to give the title compound (239.8 mg, 99% yield).

N-[(5-Chloro-2-{2-[(cis)-3-(4-fluoro-phenoxy)-8-aza-bicyclo[3.2.1]oct-8-yl]-2-oxo-ethoxy}-benzyloxy)-acetyl]-methanesulfonamide

To a solution of (5-chloro-2-{2-[(cis)-3-(4-fluoro-phenoxy)-8-aza-bicyclo[3.2.1]oct-8-yl]-2-oxo-ethoxy}-benzyloxy)-acetic acid (50 mg, 0.105 mmol) in dichlormethane (1 ml) was added 4-(dimethylamino)pyridine (19 mg, 0.157 mmol), 1-(3-dimethylaminopropyl)-3-ethylcarbodiimide hydrochloride (30 mg, 0.156 mmol), triethylamine (23 mg, 0.230 mmol) and methane sulfonamide (12 mg, 0.126 mmol). The reaction was stirred at ambient temperature overnight. The reaction was diluted with saturated aqueous sodium hydrogen carbonate and extracted with dichloromethane (3 times). The organic layers were combined, dried over sodium sulfate, filtered and concentrated in vacuo. Silica gel chromatography gave the title compound (29.3 mg, 50% yield, LRMS M+H=555.2).

The title compounds for Examples 100-102 were prepared by a method analogous to that described in Example 99.

| | | LRMS |
|---|---|---|
| Example | IUPAC name | M + H |
| 100 | (5-Chloro-2-{2-[(cis)-3-(4-fluoro-phenoxy)-8- | M − H |
| | aza-bicyclo[3.2.1]oct-8-yl]-2-oxo-ethoxy}- | 476.3 |
| | benzyloxy)-acetic acid | |
| 101 | 2-(5-Chloro-2-{2-[(cis)-3-(4-fluoro-phenoxy)-8- | M − H |
| | aza-bicyclo[3.2.1]oct-8-yl]-2-oxo-ethoxy}- | 475.3 |
| | benzyloxy)-acetamide | |
| 102 | 2-(5-Chloro-2-{2-[(cis)-3-(4-fluoro-phenoxy)-8- | 545.2 |
| | aza-bicyclo[3.2.1]oct-8-yl]-2-oxo-ethoxy}- | |
| | benzyloxy)-N-(1H-tetrazol-5-yl)-acetamide | |

### Example 103

### 2-{4-Chloro-2-[(1H-tetrazol-5-ylamino)-methyl]-phenoxy}-1-[(cis)-3-(4-fluoro-phenoxy)-8-aza-bicyclo[3.2.1]oct-8-yl]-ethanone

To a solution of 5-chloro-2-{2-[(cis)-3-(4-fluoro-phenoxy)-8-aza-bicyclo[3.2.1] oct-8-yl]-2-oxo-ethoxy}-benzaldehyde (240 mg, 0.574 mmol) in ethanol (2 ml) was added 2-amino tetrazole monohydrate (59 mg, 0.574 mmol) and acetic acid (34 mg, 0.574 mmol). The reaction was stirred for 35 minutes at ambient temperature and then refluxed for 4 hours. The reaction was cooled to ambient temperature and concentrated. The resulting residue was diluted with ethanol (3 ml) and treated with the slow addition of sodium borohydride (70 mg, 1.84 mmol). The reaction was stirred at ambient temperature for 18 hours. The reaction was concentrated, diluted with water, neutralized with 2 M aqueous hydrochloric acid and extracted with dichlormethane (3 times). The organic layers were combined, dried over sodium sulfate, filtered and concentrated in vacuo. Silica gel chromatography gave the title compound (58.8 mg, 22% yield, LRMS M+H=487.2).

### Examples 104 and 105

### 2-[2-(5-Amino-tetrazol-2-ylmethyl)-4-chloro-phenoxy]-1-[(cis)-3-(4-fluoro-phenoxy)-8-aza-bicyclo[3.2.1]oct-8-yl]-ethanone and 2-[2-(5-Amino-tetrazol-1-ylmethyl)-4-chloro-phenoxy]-1-[(cis)-3-(4-fluoro-phenoxy)-8-aza-bicyclo[3.2.1]oct-8-yl]-ethanone

2-(4-Chloro-2-chloromethyl-phenoxy)-1-[(cis)-3-(4-fluoro-phenoxy)-8-aza-bicyclo[3.2.1]oct-8-yl]-ethanone

To a solution of 2-(4-chloro-2-hydroxymethyl-phenoxy)-1-[(cis)-3-(4-fluoro-phenoxy)-8-aza-bicyclo[3.2.1]oct-8-yl]-ethanone (195 mg, 0.464 mmol) in dichloromethane (4 ml) was added thionyl chloride (66 mg, 0.557 mmol). The reaction was refluxed for two hours, cooled and concentrated. Chromatography on silica gel gave the title compound (152.3 mg, 75% yield).

2-[2-(5-Amino-tetrazol-2-ylmethyl)-4-chloro-phenoxy]-1-[(cis)-3-(4-fluoro-phenoxy)-8-aza-bicyclo[3.2.1]oct-8-yl]-ethanone and 2-[2-(5-Amino-tetrazol-1-ylmethyl)-4-chloro-phenoxy]-1-[(cis)-3-(4-fluoro-phenoxy)-8-aza-bicyclo[3.2.1]oct-8-yl]-ethanone

To a solution of 2-(4-chloro-2-chloromethyl-phenoxy)-1-[(cis)-3-(4-fluoro-phenoxy)-8-aza-bicyclo[3.2.1]oct-8-yl]-ethanone (75 mg, 0.171 mmol) in 2-butanone (1 ml) was added 2-amino tetrazole (16 mg, 0.188 mmol), potassium carbonate (47 mg, 0.342 mmol) and potassium iodide (28 mg, 0.171 mmol). The reaction was heated at 80° C. overnight. The reaction was cooled, diluted with water, and extracted with ethyl acetate (3 times). The combined organic layers were dried over sodium sulfate, filtered and concentrated in vacuo. Silica gel chromatography gave the title compounds (2-[2-(5-Amino-tetrazol-1-ylmethyl)-4-chloro-phenoxy]-1-[(cis)-3-(4-fluoro-phenoxy)-8-aza-bicyclo[3.2.1]oct-8-yl]-ethanone: 10.8 mg, 14%, LRMS M+H=487.2; 2-[2-(5-Amino-tetrazol-2-ylmethyl)-4-chloro-phenoxy]-1-[(cis)-3-(4-fluoro-phenoxy)-8-aza-bicyclo[3.2.1]oct-8-yl]-ethanone: 11.6 mg, 15%, LRMS M+H=487.2).

### Example 106

### 2-[4-Chloro-2-(1H-tetrazol-5-yl methyl)-phenoxy]-1-[(cis)-3-(4-fluoro-phenoxy)-8-aza-bicyclo[3.2.1]oct-8-yl]-ethanone

5-Chloro-2-{2-[(cis)-3-(4-fluoro-phenoxy)-8-aza-bicyclo[3.2.1]oct-8-yl]-2-oxo-ethoxy}-phenyl)-acetonitrile

To a solution of 2-(4-chloro-2-chloromethyl-phenoxy)-1-[(cis)-3-(4-fluoro-phenoxy)-8-aza-bicyclo[3.2.1]oct-8-yl]-ethanone (75 mg, 0.171 mmol) in acetonitrile (2 ml) was added sodium cyanide (17 mg, 0.342 mmol) and 18-crown-6 (5 mg, 0.017 mmol). The reaction was stirred at ambient temperature overnight. The reaction was diluted with saturated aqueous sodium bicarbonate and extracted with ethyl acetate (3 times). The organic layers were combined, dried over sodium sulfate, filtered and concentrated in vacuo. Silica gel chromatography gave the title compound (58.4 mg, 73% yield).

2-[4-Chloro-2-(1H-tetrazol-5-ylmethyl)-phenoxy]-1-1-[(cis)-3-(4-fluoro-phenoxy)-8-aza-bicyclo[3.2.1]oct-8-yl]-ethanone

To a solution of (5-chloro-2-{2-[(cis)-3-(4-fluoro-phenoxy)-8-aza-bicyclo[3.2.1]oct-8-yl]-2-oxo-ethoxy}-phenyl)-acetonitrile (58 mg, 0.135 mmol) in toluene (2 ml) was added trimethyltin azide (33 mg, 0.162 mmol). The reaction was heated at 100° C. for 36 hours. The reaction was cooled, concentrated and chromatagraphed on silica gel to give the title compound (30.4 mg, 48% yield, LRMS M+H=472.1).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of the formula 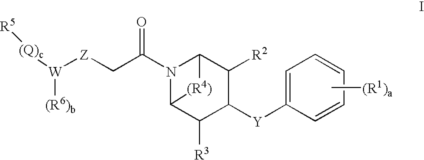
or pharmaceutically acceptable salts, tautomers, and pro-drugs thereof; wherein
a is 1, 2, 3, 4 or 5;
b is 0, 1, 2, 3, or 4;
c is 0 or 1;
Q is (C₁-C₆)alkyl;
W is (C₆-C₁₀)aryl or (C₂-C₉)heteroaryl;
Y is oxygen, or NR⁸ wherein R⁸ is hydrogen or (C₁-C₆)alkyl;
Z is oxygen or NR⁹, where R⁹ is hydrogen, (C₁-C₆)alkyl, or acetyl;
each R¹ is independently selected from the group consisting of: hydrogen, halo, cyano, nitro, trifluoromethyl, trifluoromethoxy, (C₁-C₆)alkyl, hydroxy, (C₁-C₆)alkylcarbonyloxy, and (C₁-C₆)alkoxy;
R² and R³ are each independently hydrogen or (C₁-C₆)alkyl optionally substituted with 1 to 3 halo groups;
R⁴ is (C₁-C₆)alkylene or —(CH₂)ₓ—O—(CH₂)_{y}—, wherein x and y are each independently 1 or 2;
R⁵ is selected from a list consisting of hydrogen, halo, (C₁-C₆)alkyl optionally substituted with 1 to 3 halo groups, [(C₁-C₆)alkyl]₂amino(C₁-C₆)alkylaminocarbonyl, amino(C₁-C₆)alkylaminocarbonyl, (C₁-C₆)alkylamino(C₁-C₆)alkylaminocarbonyl cyano, nitro, (C₁-C₆)alkoxy, aminocarbonyl, (C₁-C₆)alkylaminocarbonyl, [(C₁-C₆)alkyl]₂aminocarbonyl, (C₁-C₆)alkylsulfonylamino, (C₁-C₆)alkylsulfonylaminocarbonyl, ureido, aminosulfonyl, [(C₁-C₆)alkyl]₂aminosulfonyl, (C₁-C₆)alkylaminosulfonyl, [(C₁-C₆)alkyl]₂aminocarbonyl(C₁-C₆)alkylaminocarbonyl, (C₁-C₆)alkylaminocarbonyl(C₁-C₆)alkylaminocarbonyl, aminocarbonyl(C₁-C₆)alkylaminocarbonyl, (C₁-C₆)alkylsulfonylamino, hydroxy(C₁-C₆)alkylcarbonylamino, ureido(C₁-C₆)alkylaminocarbonyl, [(C₁-C₆)alkyl]₂ureido(C₁-C₆)alkylaminocarbonyl, (C₁-C₆)alkylureido(C₁-C₆)alkylaminocarbonyl, (C₂-C₉)heteroarylaminocarbonyl, carboxy, (C₁-C₆)alkoxy(C₁-C₆)alkyl(C₂-C₉)heterocyclecarbonyl, (C₂-C₉)heterocyclecarbonyl, hydroxy(C₂-C₉)heterocyclecarbonyl, aminocarbonyl(C₂-C₉)heterocyclecarbonyl, carboxy(C₂-C₉)heterocyclecarbonyl, amino(C₂-C₉)heteroaryl(C₁-C₆)alkyl, (C₁-C₆)alkylamino(C₂-C₉)heteroaryl(C₁-C₆)alkyl, [(C₁-C₆)alkyl]₂amino(C₂-C₉)heteroaryl(C₁-C₆)alkyl, (C₂-C₉)heteroarylamino(C₁-C₆)alkyl, (C₂-C₉)heteroarylaminocarbonyl(C₁-C₆)alkoxy, (C₁-C₆)alkylsulfonylaminocarbonyl(C₁-C₆)alkoxy, aminocarbonyl(C₁-C₆)alkoxy, carboxy(C₁-C₆)alkoxy, aminosulfonyl, (C₁-C₆)alkylcarbonylaminosulfonyl, hydroxy(C₁-C₆)alkylcarbonylaminosulfonyl, (C₁-C₆)alkoxycarbonylaminosulfonyl, (C₁-C₆)alkoxy(C₁-C₆)alkylcarbonylaminosulfonyl, hydroxysulfonyl, hydroxysulfonyl(C₁-C₆)alkylcarbonylthiol, carboxy(C₁-C₆)alkylthiol hydroxy, hydroxy(C₁-C₆)alkylaminocarbonyl, carboxy(C₂-C₉)heterocycloxy or [carboxy][amino](C₁-C₆)alkoxy, aminocarbonyl(C₁-C₆)alkylcarbonylamino, (C₁-C₆)alkylaminocarbonyl(C₁-C₆)alkylcarbonylamino, [(C₁-C₆)alkyl]₂aminocarbonyl(C₁-C₆)alkylcarbonylamino, amino(C₁-C₆)alkylcarbonylamino, (C₁-C₆)alkylamino(C₁-C₆)alkylcarbonylamino, [(C₁-C₆)alkyl]₂amino(C₁-C₆)alkylcarbonylamino, ureido(C₁-C₆)alkylcarbonylamino, (C₁-C₆)alkylureido(C₁-C₆)alkylcarbonylamino, [(C₁-C₆)alkyl]₂ureido(C₁-C₆)alkylcarbonylamino, amino(C₁-C₆)alkylsulfonylamino, amino(C₁-C₆)alkylcarbonylaminosulfonyl, (C₁-C₆)alkylamino(C₁-C₆)alkylcarbonylaminosulfonyl, [(C₁-C₆)alkyl]₂amino(C₁-C₆)alkylcarbonylaminosulfonyl, aminosulfonylamino, (C₁-C₆)alkylaminosulfonylamino, [(C₁-C₆)alkyl]₂aminosulfonylamino, (C₂-C₉)heterocycloxy, (C₂-C₉)heteroaryloxy, (C₂-C₉)heterocycleamino, (C₂-C₉)heteroarylamino, amino, (C₁-C₆)alkylamino, [(C₁-C₆)alkyl]₂amino, amino(C₁-C₆)alkoxy, (C₁-C₆)alkylamino(C₁-C₆)alkoxy, [(C₁-C₆)alkyl]₂amino(C₁-C₆)alkoxy, amino(C₁-C₆)alkylamino, (C₁-C₆)alkylcarbonylamino(C₁-C₆)alkylamino, ureido(C₁-C₆)alkylamino, hydroxy(C₁-C₆)alkylamino, (C₁-C₆)alkoxy(C₁-C₆)alkylamino, and (C₁-C₆)alkylsulfonylamino(C₁-C₆)alkylamino;
each R⁶ is independently selected from a list consisting of: hydrogen, halo, (C₁-C₆)alkyl optionally substituted with 1 to 3 halo groups; cyano, (C₁-C₆)alkoxy, aminocarbonyl, carboxy, nitro, (C₁-C₆)alkylcarbonyl, and (C₁-C₆)alkoxy optionally substituted by 1 to 3 halo groups.

2. A compound according to claim 1, wherein R¹ is halo; a is 1 or 2; Y is oxygen; Z is oxygen; R⁴ is a —CH₂—CH₂— diradical; R⁴ is ‘cis’ to the Y group; R² and R³ are each hydrogen; W is phenyl; b is 0, 1 or 2, and R⁶ is selected from the group consisting of halo, (C₁-C₆)alkyl, cyano, and (C₁-C₆)alkylcarbonyl.

3. A compound according to claim 1, wherein R¹ is halo; a is 1 or 2; Y is oxygen; Z is oxygen or NH; R⁴ is a —CH₂—CH₂— diradical; R⁴ is ‘cis’ to the Y group, R² and R³ are each hydrogen; W is pyridyl; b is 0, 1 or 2, and R⁶ is selected from the group consisting of halo, (C₁-C₆)alkyl, cyano, and (C₁-C₆)alkylcarbonyl.

4. A compound according to claim 1, wherein c is 0, and R⁵ is selected from the group consisting of aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, and carboxy(C₁-C₆)alkoxy.

5. A compound according to claim 1, wherein c is 1, and R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, and carboxy.

6. A compound according to claim 2, wherein c is 0; R⁵ is selected from the group consisting of aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy; and R⁶ is selected from the group consisting of halo, (C₁-C₆)alkyl, cyano, and (C₁-C₆)alkylcarbonyl.

7. A compound according to claim 3, wherein c is 0; R⁵ is selected from the group consisting of: aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy; and R⁶ is selected from the group consisting of halo, (C₁-C₆)alkyl, cyano, and (C₁-C₆)alkylcarbonyl.

8. A compound according to claim 2, wherein c is 1; R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy; and R⁶ is selected from the group consisting of halo, (C₁-C₆)alkyl, cyano, and (C₁-C₆)alkylcarbonyl.

9. A compound according to claim 3, wherein c is 1; R⁵ is selected from the group consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy; and R⁶ is selected from the group consisting of halo, (C₁-C₆)alkyl, cyano, and (C₁-C₆)alkylcarbonyl.

10. A compound according to claim 1, wherein said compound is selected from the group consisting of:
5-Chloro-2-{2-[(trans)-3-(4-fluoro-phenoxy)-8-aza-bicyclo[3.2.1]oct-8-yl]-2-oxo-ethoxy}-benzamide;
5-Chloro-2-{2-[(cis)-3-(4-fluoro-phenoxy)-8-aza-bicyclo[3.2.1]oct-8-yl]-2-oxo-ethoxy}-benzamide;
2-{2-[(cis)-3-(4-Fluoro-phenoxy)-8-aza-bicyclo[3.2.1]oct-8-yl]-2-oxo-ethoxy}-4-methoxy-benzamide;
5-Chloro-2-{2-[(cis)-3-(4-fluoro-phenoxy)-8-aza-bicyclo[3.2.1]oct-8-yl]-2-oxo-ethoxy}-benzenesulfonamide;
N-Carbamoylmethyl-5-chloro-2-{2-[(cis)-3-(4-fluoro-phenoxy)-8-aza-bicyclo[3.2.1]oct-8-yl]-2-oxo-ethoxy}-benzamide;
(5-Chloro-2-{2-[(cis)-3-(4-fluoro-phenoxy)-8-aza-bicyclo[3.2.1]oct-8-yl]-2-oxo-ethoxy}-benzoylamino)-acetic acid;
N-(5-Chloro-2-{2-[(cis)-3-(4-fluoro-phenoxy)-8-aza-bicyclo[3.2.1]oct-8-yl]-2-oxo-ethoxy}-phenyl)-methanesulfonamide;
N-(5-Chloro-2-{2-[(cis)-3-(4-fluoro-phenoxy)-8-aza-bicyclo[3.2.1]oct-8-yl]-2-oxo-ethoxy}-phenyl)-3-hydroxy-3-methyl-butyramide;
(5-Chloro-2-{2-[(cis)-3-(4-fluoro-phenoxy)-8-aza-bicyclo[3.2.1]oct-8-yl]-2-oxo-ethoxy}-phenyl)-urea;
(5-Chloro-2-{2-[(trans)-3-(4-fluoro-phenoxy)-8-aza-bicyclo[3.2.1]oct-8-yl]-2-oxo-ethoxy}-phenyl)-urea;
5-Chloro-2-{2-[(cis)-3-(4-fluoro-phenoxy)-8-aza-bicyclo[3.2.1]oct-8-yl]-2-oxo-ethoxy}-N-(2-ureido-ethyl)-benzamide;
5-Chloro-2-{2-[(cis)-3-(4-fluoro-phenoxy)-8-aza-bicyclo[3.2.1]oct-8-yl]-2-oxo-ethoxy}-N-(2H-tetrazol-5-yl)-benzamide;
5-Chloro-2-{2-[(cis)-3-(4-fluoro-phenoxy)-8-aza-bicyclo[3.2.1]oct-8-yl]-2-oxo-ethoxy}-benzoic acid;
5-Chloro-2-{2-[(cis)-3-(4-fluoro-phenoxy)-8-aza-bicyclo[3.2.1]oct-8-yl]-2-oxo-ethoxy}-N-pyridin-2-yl-benzamide;
2-[4-Chloro-2-((2R)-2-methoxymethyl-pyrrolidine-1-carbonyl)-phenoxy]-1-[(cis)-3-(4-fluoro-phenoxy)-8-aza-bicyclo[3.2.1]oct-8-yl]-ethanone;
2-[4-Chloro-2-(morpholine-4-carbonyl)-phenoxy]-1-(cis)-[3-(4-fluoro-phenoxy)-8-aza-bicyclo[3.2.1]oct-8-yl]-ethanone;
N-(2-{2-[3-(4-Fluoro-phenoxy)-8-aza-bicyclo[3.2.1]oct-8-yl]-2-oxo-ethoxy}-5-trifluoromethyl-phenyl)-methanesulfonamide;
5-Chloro-N-(2-dimethylamino-ethyl)-2-{2-[(cis)-3-(4-fluoro-phenoxy)-8-aza-bicyclo[3.2.1]oct-8-yl]-2-oxo-ethoxy}-benzamide;
2-[4-Chloro-2-((3S)-3-hydroxy-pyrrolidine-1-carbonyl)-phenoxy]-1-[(cis)-3-(4-fluoro-phenoxy)-8-aza-bicyclo[3.2.1]oct-8-yl]-ethanone;
2-[4-Chloro-2-((2S)-2-methoxymethyl-pyrrolidine-1-carbonyl)-phenoxy]-1-[(cis)-3-(4-fluoro-phenoxy)-8-aza-bicyclo[3.2.1]oct-8-yl]-ethanone;
2-[4-Chloro-2-((3R)-3-hydroxy-pyrrolidine-1-carbonyl)-phenoxy]-1-[(cis)-3-(4-fluoro-phenoxy)-8-aza-bicyclo[3.2.1]oct-8-yl]-ethanone;
1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2S)-2-carboxylic acid;
N-(2-Amino-ethyl)-5-chloro-2-{2-[3-(4-fluoro-phenoxy)-8-aza-bicyclo[3.2.1]oct-8-yl]-2-oxo-ethoxy}-benzamide;
1-(5-Chloro-2-{2-[(cis)-3-(4-fluoro-phenoxy)-8-aza-bicyclo[3.2.1]oct-8-yl]-2-oxo-ethoxy}-benzoyl)-(4S)-4-hydroxy-pyrrolidine-(2S)-2-carboxylic acid amide;
1-(5-Chloro-2-{2-[(cis)-3-(4-fluoro-phenoxy)-8-aza-bicyclo[3.2.1]oct-8-yl]-2-oxo-ethoxy}-benzoyl)-(4S)-4-hydroxy-pyrrolidine-(2S)-2-carboxylic acid;
1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2S)-2-carboxylic acid amide;
1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2R)-2-carboxylic acid amide;
1-(5-Chloro-2-{2-[(cis)-3-(4-fluoro-phenoxy)-8-aza-bicyclo[3.2.1]oct-8-yl]-2-oxo-ethoxy}-benzoyl)-(4R)-4-hydroxy-pyrrolidine-(2R)-2-carboxylic acid;
2-(5-Chloro-quinolin-8-yloxy)-1-[(cis)-3-(4-fluoro-phenoxy)-8-aza-bicyclo[3.2.1]oct-8-yl]-ethanone;
(5-Chloro-2-{2-[(cis)-3-(4-fluoro-phenoxy)-8-aza-bicyclo[3.2.1]oct-8-yl]-2-oxo-ethoxy}-phenyl)-acetic acid;
5-Chloro-2-{2-[(trans)-7-(4-fluoro-phenoxy)-3-oxa-9-aza-bicyclo[3.3.1]non-9-yl]-2-oxo-ethoxy}-benzamide;
2-(5-Chloro-2-{2-[(cis)-3-(4-fluoro-phenoxy)-8-aza-bicyclo[3.2.1]oct-8-yl]-2-oxo-ethoxy}-phenyl)-acetamide;
N-(5-Chloro-2-{2-[(cis)-3-(4-fluoro-phenoxy)-8-aza-bicyclo[3.2.1]oct-8-yl]-2-oxo-ethoxy}-benzoyl)-methanesulfonamide;
N-[(5-Chloro-2-{2-[(cis)-3-(4-fluoro-phenoxy)-8-aza-bicyclo[3.2.1]oct-8-yl]-2-oxo-ethoxy}-phenyl)-acetyl]-methanesulfonamide;
2-[2-(5-Amino-tetrazol-1-ylmethyl)-4-chloro-phenoxy]-1-[(cis)-3-(4-fluoro-phenoxy)-8-aza-bicyclo[3.2.1]oct-8-yl]-ethanone;
2-[2-(5-Amino-tetrazol-2-ylmethyl)-4-chloro-phenoxy]-1-[(cis)-3-(4-fluoro-phenoxy)-8-aza-bicyclo[3.2.1]oct-8-yl]-ethanone;
5-Chloro-2-{2-[(cis)-3-(4-fluoro-phenoxy)-8-aza-bicyclo[3.2.1]oct-8-yl]-2-oxo-ethoxy}-N-pyrimidin-4-yl-benzamide;
2-[4-Chloro-2-(1H-tetrazol-5-yl)-phenoxy]-1-[(cis)-3-(4-fluoro-phenoxy)-8-aza-bicyclo[3.2.1]oct-8-yl]-ethanone;
2-[4-Chloro-2-(1H-tetrazol-5-ylmethyl)-phenoxy]-1-[(cis)-3-(4-fluoro-phenoxy)-8-aza-bicyclo[3.2.1]oct-8-yl]-ethanone;
(5-Chloro-2-{2-[(trans)-3-(4-fluoro-phenoxy)-8-aza-bicyclo[3.2.1]oct-8-yl]-2-oxo-ethoxy}-phenyl)-acetic acid;
N-[(5-Chloro-2-{2-[(trans)-3-(4-fluoro-phenoxy)-8-aza-bicyclo[3.2.1]oct-8-yl]-2-oxo-ethoxy}-phenyl)-acetyl]-methanesulfonamide;
2-(5-Chloro-2-{2-[(trans)-3-(4-fluoro-phenoxy)-8-aza-bicyclo[3.2.1]oct-8-yl]-2-oxo-ethoxy}-phenyl)-acetamide;
2-{4-Chloro-2-[(1H-tetrazol-5-ylamino)-methyl]-phenoxy}-1-[(cis)-3-(4-fluoro-phenoxy)-8-aza-bicyclo[3.2.1]oct-8-yl]-ethanone;
(5-Chloro-2-{2-[(trans)-7-(4-fluoro-phenoxy)-3-oxa-9-aza-bicyclo[3.3.1]non-9-yl]-2-oxo-ethoxy}-phenyl)-acetic acid;
2-[4-Chloro-2-(1-hydroxy-1-methyl-ethyl)-phenoxy]-1-(cis)-[3-(4-fluoro-phenoxy)-8-aza-bicyclo[3.2.1]oct-8-yl]-ethanone;
N-[(5-Chloro-2-{2-[(trans)-7-(4-fluoro-phenoxy)-3-oxa-9-aza-bicyclo[3.3.1]non-9-yl]-2-oxo-ethoxy}-phenyl)-acetyl]-methanesulfonamide;
(5-Chloro-2-{2-[(cis)-3-(4-fluoro-phenoxy)-8-aza-bicyclo[3.2.1]oct-8-yl]-2-oxo-ethoxy}-benzyloxy)-acetic acid;
2-(5-Chloro-2-{2-[(cis)-3-(4-fluoro-phenoxy)-8-aza-bicyclo[3.2.1]oct-8-yl]-2-oxo-ethoxy}-benzyloxy)-N-(1H-tetrazol-5-yl)-acetamide;
N-[(5-Chloro-2-{2-[(cis)-3-(4-fluoro-phenoxy)-8-aza-bicyclo[3.2.1]oct-8-yl]-2-oxo-ethoxy}-benzyloxy)-acetyl]-methanesulfonamide;
2-(5-Chloro-2-{2-[(cis)-3-(4-fluoro-phenoxy)-8-aza-bicyclo[3.2.1]oct-8-yl]-2-oxo-ethoxy}-benzyloxy)-acetamide;
(5-Bromo-2-{2-[(cis)-3-(4-fluoro-phenoxy)-8-aza-bicyclo[3.2.1]oct-8-yl]-2-oxo-ethoxy}phenyl)-acetic acid;
2-(5-Bromo-2-{2-[(cis)-3-(4-fluoro-phenoxy)-8-aza-bicyclo[3.2.1]oct-8-yl]-2-oxo-ethoxy}-phenyl)-acetamide;
N-[(5-Bromo-2-{2-[(cis)-3-(4-fluoro-phenoxy)-8-aza-bicyclo[3.2.1]oct-8-yl]-2-oxo-ethoxy}-phenyl)-acetyl]-methanesulfonamide;
(5-Chloro-2-{2-[(cis)-3-(4-fluoro-phenoxy)-8-aza-bicyclo[3.2.1]oct-8-yl]-2-oxo-ethoxy}-phenyl)-methanesulfonamide;
N-Acetyl-C-(5-chloro-2-{2-[(cis)-3-(4-fluoro-phenoxy)-8-aza-bicyclo[3.2.1]oct-8-yl]-2-oxo-ethoxy}-phenyl)-methanesulfonamide;
(5-Bromo-2-{2-[(cis)-3-(4-fluoro-phenoxy)-8-aza-bicyclo[3.2.1]oct-8-yl]-2-oxo-ethoxy}-phenyl)-methanesulfonamide;
N-Acetyl-C-(5-bromo-2-{2-[(cis)-3-(4-fluoro-phenoxy)-8-aza-bicyclo[3.2.1]oct-8-yl]-2-oxo-ethoxy}-phenyl)-methanesulfonamide;
C-(5-Chloro-2-{2-[(cis)-3-(4-fluoro-phenoxy)-8-aza-bicyclo[3.2.1]oct-8-yl]-2-oxo-ethoxy}-phenyl)-N-(2-hydroxy-2-methyl-propionyl)-methanesulfonamide;
C-(5-Chloro-2-{2-[(cis)-3-(4-fluoro-phenoxy)-8-aza-bicyclo[3.2.1]oct-8-yl]-2-oxo-ethoxy}-phenyl)-N-hydroxyacetyl-methanesulfonamide;
C-(5-Chloro-2-{2-[(cis)-3-(4-fluoro-phenoxy)-8-aza-bicyclo[3.2.1]oct-8-yl]-2-oxo-ethoxy}-phenyl)-N-(methoxycarbonyl)-methanesulfonamide;
3-(5-Chloro-2-{2-[(cis)-3-(4-fluoro-phenoxy)-8-aza-bicyclo[3.2.1]oct-8-yl]-2-oxo-ethoxy}-phenyl)-propionic acid;
C-(5-Chloro-2-{2-[(cis)-3-(4-fluoro-phenoxy)-8-aza-bicyclo[3.2.1]oct-8-yl]-2-oxo-ethoxy}-phenyl)-N-(1-hydroxy-cyclopropanecarbonyl)-methanesulfonamide;
N-[3-(5-Chloro-2-{2-[(cis)-3-(4-fluoro-phenoxy)-8-aza-bicyclo[3.2.1]oct-8-yl]-2-oxo-ethoxy}-phenyl)-propionyl]-methanesulfonamide;
C-(5-Chloro-2-{2-[(cis)-3-(4-fluoro-phenoxy)-8-aza-bicyclo[3.2.1]oct-8-yl]-2-oxo-ethoxy}-phenyl)-N-methoxyacetyl-methanesulfonamide;
4-{2-[(cis)-3-(4-Fluoro-phenoxy)-8-aza-bicyclo[3.2.1]oct-8-yl]-2-oxo-ethoxy}-benzoic acid;
1-[(cis)-3-(4-Fluoro-phenoxy)-8-aza-bicyclo[3.2.1]oct-8-yl]-2-phenoxy-ethanone;
2-(4-Bromo-phenoxy)-1-[(cis)-3-(4-fluoro-phenoxy)-8-aza-bicyclo[3.2.1]oct-8-yl]-ethanone;
1-[(cis)-3-(4-Fluoro-phenoxy)-8-aza-bicyclo[3.2.1]oct-8-yl]-2-(4-trifluoromethyl-phenoxy)-ethanone;
1-[(cis)-3-(4-Fluoro-phenoxy)-8-aza-bicyclo[3.2.1]oct-8-yl]-2-p-tolyloxy-ethanone;
2-(4-Chloro-phenoxy)-1-(cis)-[3-(4-fluoro-phenoxy)-8-aza-bicyclo[3.2.1]oct-8-yl]-ethanone;
(5-Chloro-2-{2-[(cis)-3-(4-fluoro-phenoxy)-8-aza-bicyclo[3.2.1]oct-8-yl]-2-oxo-ethoxy}-phenyl)-methanesulfonic acid;
2-(2-Acetyl-4-chloro-phenoxy)-1-[(cis)-3-(4-fluoro-phenoxy)-8-aza-bicyclo[3.2.1]oct-8-yl]-ethanone;
5-Chloro-2-{2-[(cis)-3-(4-fluoro-phenoxy)-8-aza-bicyclo[3.2.1]oct-8-yl]-2-oxo-ethoxy}-N-methyl-benzamide;
5-Bromo-2-{2-[(cis)-3-(4-fluoro-phenoxy)-8-aza-bicyclo[3.2.1]oct-8-yl]-2-oxo-ethoxy}-benzamide;
2-(4-Chloro-2-hydroxymethyl-phenoxy)-1-[(cis)-3-(4-fluoro-phenoxy)-8-aza-bicyclo[3.2.1]oct-8-yl]-ethanone;
2-(4-Bromo-2-hydroxymethyl-phenoxy)-1-(cis)-[3-(4-fluoro-phenoxy)-8-aza-bicyclo[3.2.1]oct-8-yl]-ethanone;
2-(4-Chloro-2-hydroxy-phenoxy)-1-[(cis)-3-(4-fluoro-phenoxy)-8-aza-bicyclo[3.2.1]oct-8-yl]-ethanone;
(5-Chloro-2-{2-[(cis)-3-(4-fluoro-phenoxy)-8-aza-bicyclo[3.2.1]oct-8-yl]-2-oxo-ethoxy}-phenoxy)-acetic acid;
2-(4-Bromo-2-hydroxy-phenoxy)-1-[(cis)-3-(4-fluoro-phenoxy)-8-aza-bicyclo[3.2.1]oct-8-yl]-ethanone;
5-Chloro-2-{2-[(cis)-3-(4-fluoro-phenoxy)-8-aza-bicyclo[3.2.1]oct-8-yl]-2-oxo-ethoxy}-N-(2-hydroxy-ethyl)-benzamide;
5-Chloro-2-{2-[(cis)-3-(4-fluoro-phenoxy)-8-aza-bicyclo[3.2.1]oct-8-yl]-2-oxo-ethoxy}-N-(3-hydroxy-propyl)-benzamide;
4-(5-Chloro-2-{2-[(cis)-3-(4-fluoro-phenoxy)-8-aza-bicyclo[3.2.1]oct-8-yl]-2-oxo-ethoxy}-phenoxy)-pyrrolidine-(2S)-2-carboxylic acid;
(2S)-2-Amino-4-(5-chloro-2-{2-[(cis)-3-(4-fluoro-phenoxy)-8-aza-bicyclo[3.2.1]oct-8-yl]-2-oxo-ethoxy}-phenoxy)-butyric acid;
(cis)-5-Chloro-2-{2-[3-(4-fluoro-phenoxy)-8-aza-bicyclo[3.2.1]oct-8-yl]-2-oxo-ethylamino}-nicotinic acid;
5-Chloro-2-{2-[3-(4-fluoro-phenoxy)-8-aza-bicyclo[3.2.1]oct-8-yl]-2-oxo-ethylamino}-nicotinamide;
(cis)-5-Chloro-N-(2-dimethylamino-ethyl)-2-{2-[3-(4-fluoro-phenoxy)-8-aza-bicyclo[3.2.1]oct-8-yl]-2-oxo-ethylamino}-nicotinamide;
(cis)-N-(2-Amino-ethyl)-5-chloro-2-{2-[3-(4-fluoro-phenoxy)-8-aza-bicyclo[3.2.1]oct-8-yl]-2-oxo-ethylamino}-nicotinamide;
[(cis)-(5-Chloro-2-{2-[3-(4-fluoro-phenoxy)-8-aza-bicyclo[3.2.1]oct-8-yl]-2-oxo-ethylamino}-pyridine-3-carbonyl)-amino]-acetic acid;
2-[5-Chloro-3-(morpholine-4-carbonyl)-pyridin-2-ylamino]-1-[(cis)-3-(4-fluoro-phenoxy)-8-aza-bicyclo[3.2.1]oct-8-yl]-ethanone;
2-[5-Chloro-3-((3S)-3-hydroxy-pyrrolidine-1-carbonyl)-pyridin-2-ylamino]-1-[(cis)-3-(4-fluoro-phenoxy)-8-aza-bicyclo[3.2.1]oct-8-yl]-ethanone;
2-[5-Chloro-3-((3R)-3-hydroxy-pyrrolidine-1-carbonyl)-pyridin-2-ylamino]-1-[(cis)-3-(4-fluoro-phenoxy)-8-aza-bicyclo[3.2.1]oct-8-yl]-ethanone;
2-[5-Chloro-3-((2S)-2-methoxymethyl-pyrrolidine-1-carbonyl)-pyridin-2-ylamino]-1-[(cis)-3-(4-fluoro-phenoxy)-8-aza-bicyclo[3.2.1]oct-8-yl]-ethanone;
2-[5-Chloro-3-((2R)-2-methoxymethyl-pyrrolidine-1-carbonyl)-pyridin-2-ylamino]-1-[(cis)-3-(4-fluoro-phenoxy)-8-aza-bicyclo[3.2.1]oct-8-yl]-ethanone;
(cis)-N-Carbamoylmethyl-5-chloro-2-{2-[3-(4-fluoro-phenoxy)-8-aza-bicyclo[3.2.1]oct-8-yl]-2-oxo-ethylamino}-nicotinamide;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2S)-2-carboxylic acid amide;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4S)-4-hydroxy-pyrrolidine-(2S)-2-carboxylic acid amide;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2R)-2-carboxylic acid amide;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2S)-2-carboxylic acid;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4S)-4-hydroxy-pyrrolidine-(2S)-2-carboxylic acid;
1-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-(4R)-4-hydroxy-pyrrolidine-(2R)-2-carboxylic acid;
5-Chloro-2-{2-[(cis)-3-(4-fluoro-phenoxy)-8-aza-bicyclo[3.2.1]oct-8-yl]-2-oxo-ethylamino}-N-pyrimidin-4-yl-nicotinamide;
N-(5-Chloro-2-{2-[(cis)-3-(4-fluoro-phenoxy)-8-aza-bicyclo[3.2.1]oct-8-yl]-2-oxo-ethylamino}-pyridine-3-carbonyl)-methanesulfonamide;
5-Chloro-2-{2-[(cis)-3-(4-fluoro-phenoxy)-8-aza-bicyclo[3.2.1]oct-8-yl]-2oxo-ethylamino}-N-pyridin-2-yl-nicotinamide;
5-Chloro-2-{2-[(cis)-3-(4-fluoro-phenoxy)-8-aza-bicyclo[3.2.1]oct-8-yl]-2-oxo-ethoxy}-nicotinamide;
2-(3-Amino-5-chloro-pyridin-2-yloxy)-1-[(cis)-3-(4-fluoro-phenoxy)-8-aza-bicyclo[3.2.1]oct-8-yl]-ethanone;
(5-Chloro-2-{2-[(cis)-3-(4-fluoro-phenoxy)-8-aza-bicyclo[3.2.1]oct-8-yl]-2-oxo-ethoxy}-pyridin-3-yl)-urea;
2-Amino-N-(5-chloro-2-{2-[(cis)-3-(4-fluoro-phenoxy)-8-aza-bicyclo[3.2.1]oct-8-yl]-2-oxo-ethoxy}-pyridin-3-yl)-acetamide;
N-(5-Chloro-2-{2-[(cis)-3-(4-fluoro-phenoxy)-8-aza-bicyclo[3.2.1]oct-8-yl]-2-oxo-ethoxy}-pyridin-3-yl)-succinamic acid; and
N-Acetyl-5-chloro-2-{2-[(cis)-3-(4-fluoro-phenoxy)-8-aza-bicyclo[3.2.1]oct-8-yl]-2-oxo-ethoxy}-nicotinamide.

11. A pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, comprising an amount of a compound according to claim 1, or a pharmaceutically acceptable salt thereof, that is effective in treating or preventing such disorder or condition and a pharmaceutically acceptable carrier.

12. A pharmaceutical composition for treating or preventing a disorder or condition that can be treated or prevented by inhibiting MIP-1α and/or RANTES binding to the receptor CCR1 in a mammal, comprising an amount of a compound according to claim 1, or a pharmaceutically acceptable salt thereof, effective in treating or preventing such disorder or condition and a pharmaceutically acceptable carrier.

13. A method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, comprising administering to a mammal in need of such treatment or prevention an amount of a compound according to claim 1, or a pharmaceutically acceptable salt thereof, that is effective in treating or preventing such disorder or condition.

14. A method for treating or preventing a disorder or condition that can be treated or prevented by antagonizing the CCR1 receptor in a mammal, comprising administering to a mammal in need of such treatment or prevention an amount of a compound according to claim 1, or a pharmaceutically acceptable salt thereof, that is effective in treating or preventing such disorder or condition.
